(19) United States
(12) Patent Application Publication
(10) Pub. No.: US 2021/0087156 A1
(43) Pub. Date: Mar. 25, 2021

(54) PHARMACEUTICALLY ACCEPTABLE SALTS OF BENZODICYCLOALKANE DERIVATIVE, POLYMORPHIC SUBSTANCE THEREOF, AND APPLICATION THEREOF

(71) Applicants: SHANGHAI HAIYAN PHARMACEUTICAL TECHNOLOGY CO., LTD., Shanghai (CN); YANGTZE RIVER PHARMACEUTICAL GROUP CO., LTD., Jiangsu (CN)

(72) Inventors: Zhaoling DAN, Shanghai (CN); Taotao JIANG, Shanghai (CN); Jibiao WANG, Shanghai (CN); Keyi ZHU, Shanghai (CN)

(21) Appl. No.: 17/050,375

(22) PCT Filed: Apr. 23, 2019
(86) PCT No.: PCT/CN2019/083972

§ 371 Date: Oct. 23, 2020

(30) Foreign Application Priority Data
Apr. 24, 2018 (CN) 201810372376.3

(51) Int. Cl. C07D 305/08

(52) U.S. Cl. CPC C07D 305/08; C07B 2200/13

(57) ABSTRACT

The present invention provides a pharmaceutically acceptable salt of benzodicycloalkane derivative and a polymorph thereof, and an application thereof. Specifically, the present invention provides a polymorph of benzobicyclic alkane derivative or a pharmaceutically acceptable salt thereof, and an application thereof. Furthermore, the present invention discloses a pharmaceutical composition of the compound and an application thereof.

### TECHNICAL FIELD

The present invention relates to a field of medical technology, in particular a pharmaceutically acceptable salt of benzobicyclic alkane derivative and a polymorph thereof and an application thereof.

### BACKGROUND

Pain is the most common and most confusing symptom clinically, especially for patients with postoperative, chronic pain or cancer. At present, postoperative analgesia is still dominated by pure opioid analgesics. Complications such as respiratory depression, nausea, vomiting, and itching of the skin are accompanied by a high incidence, which adds new troubles to patients with postoperative analgesia.

In recent years, dezocine has been widely used as a new type of opioid receptor mixed agonist-antagonist at home and abroad, and its analgesic effect is good and adverse reactions are few. Dezocine is a synthetic compound having benzodicycloalkane structure and a mixed opioid receptor agonist-antagonist that reduces the incidence of respiratory depression and addiction, and the activity of dezocine on δ opioid receptors is very weak and does not produce irritability and anxiety. Therefore, it is widely used in clinical postoperative analgesia.

However, one of the major disadvantages of dezocine is its poor oral bioavailability (not higher than 5%), which results in the current use of dezocine as an injection form, and another disadvantage of dezocine is its small administration window that the effect is not obvious at low doses and gradually increases with the dose, but when the effect is enhanced, the risk of adverse reactions is significantly increased. Therefore, in order to ensure the smoothness of the administration concentration, it is basically perfused in clinical practice. The injection is not only inconvenient to use, but also its onset time is short. After about 2-3 hours, the blood drug concentration falls below the effective level, and the drug effect disappears. In addition, due to the rapid elimination of blood drugs, large doses have clinically increased the risk of adverse reactions such as respiratory depression, nausea, vomiting, and itching of the skin.

Therefore, it is necessary to develop novel dezocine analogues to improve oral bioavailability, prolong the onset time, maintain a constant blood concentration, reduce clinical adverse reactions, and provide better drug selection and better compliance for clinical patients, which is of great significance. The present invention has developed various salt forms and crystal forms of dezocine analogs based on the foregoing work, which contributes to further drug development.

### SUMMARY OF THE INVENTION

It is an object of the present invention to provide a pharmaceutically acceptable salt of benzodicycloalkane derivative and a polymorph thereof and an application thereof.

In the first aspect of the present invention there is provided a pharmaceutically acceptable salt of a compound of formula X, a polymorph of the compound of formula X, or a polymorph of the pharmaceutically acceptable salt of the compound of formula X:

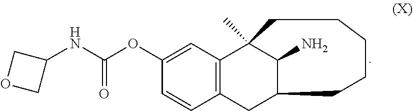 [SMILES: C[C@@]12CCCCC[C@@H](Cc3ccc(OC(=O)NC4COC4)cc31)[C@@H]2N]

In another preferred embodiment, the pharmaceutically acceptable salt is selected from the group consisting of hydrochloride, sulfate, hydrobromide, phosphate, methanesulfonate, maleate, L-tartrate, citrate, fumarate, succinate, and besylate.

In another preferred embodiment, the pharmaceutically acceptable salt of a compound of formula X or the polymorph of a compound of formula X or a pharmaceutically acceptable salt thereof is an anhydrous form, a hydrate form or a solvate form.

In another preferred embodiment, the pharmaceutically acceptable salt is selected from the group consisting of sulfate, maleate, and L-tartrate.

In another preferred embodiment, the pharmaceutically acceptable salt is maleate, and the mole ratio of maleic acid to a compound of formula X is (0.8-2.1):1, preferably 1:1.

In another preferred embodiment, the pharmaceutically acceptable salt is L-tartrate, and the mole ratio of L-tartaric acid to a compound of formula X is (0.8-2.1):1, preferably 1:1.

In another preferred embodiment, the polymorph is A-type crystal of the maleate of a compound of formula X, i.e., crystal form A, the X-ray powder diffraction pattern of which has peaks at diffraction angles 2θ (°) of the following group A1: 7.75±0.2, 11.41±0.2, 13.03±0.2, 13.66±0.2, 15.10±0.2, 18.85±0.2, 21.49±0.2, 23.98±0.2, 25.93±0.2.

In another preferred embodiment, the X-ray powder diffraction pattern of the crystal form A further has peaks at 2 or more than 2 of diffraction angles 2θ (°) selected from the following group A2: 10.66±0.2, 12.43±0.2, 15.55±0.2, 16.84±0.2, 17.92±0.2, 20.17±0.2, 27.40±0.2.

In another preferred embodiment, the X-ray powder diffraction pattern of the crystal form A further has peaks at 2 or more than 2 of diffraction angles 2θ (°) selected from the following group A3: 1.04±0.2, 14.29±0.2, 22.90±0.2, 25.15±0.2, 28.49±0.2, 28.84±0.2, 30.60±0.2, 31.57±0.2, 33.40±0.2, 37.85±0.2.

In another preferred embodiment, the X-ray powder diffraction pattern of the crystal form A has peaks at 6 or more or all (such as 6, 7, 8, 9, 10, 11, 12, 13, 14, 15, etc.) of diffraction angles 2θ (°) selected from the following groups A1, A2 and A3.

In another preferred embodiment, the X-ray powder diffraction pattern of the crystal form A has peaks at diffraction angles 2θ (°) of table A and the intensity of each peak is shown as in table A:

TABLE A

| 2θ(°) | I/I₀ | 2θ(°) | I/I₀ | 2θ(°) | I/I₀ |
|---|---|---|---|---|---|
| 1.04 | W | 7.75 | S | 10.66 | M |
| 11.41 | VS | 12.43 | M | 13.03 | S |
| 13.66 | S | 14.29 | W | 15.10 | VS |
| 15.55 | M | 16.84 | M | 17.92 | M |
| 18.85 | S | 20.17 | M | 21.49 | VS |
| 22.90 | W | 23.98 | VS | 25.15 | W |
| 25.93 | S | 27.40 | M | 28.49 | W |
| 28.84 | W | 30.60 | W | 31.57 | W |
| 33.40 | W | 37.85 | W | | |

In another preferred embodiment, the X-ray powder diffraction pattern of the crystal form A is basically shown as FIG. 1.

In another preferred embodiment, in the crystal form A, the mole ratio of maleic acid to a compound of formula X is (0.8-2.1):1, preferably (1.0-1.2):1, more preferably 1:1.

In another preferred embodiment, the crystal form A has an exothermic peak at 198.32° C. (as FIG. 2), and a degradation of 4.07% from 100° C. to 192.82° C. and a degradation of 18.90% from 192.82° C. to 295.11° C. (as FIG. 3).

In another preferred embodiment, the polymorph is B-type crystal of the sulfate of a compound of formula X, i.e., crystal form B, the X-ray powder diffraction pattern of which has peaks at diffraction angles 2θ (°) of the following group B1: 7.66±0.2, 13.57±0.2, 15.36±0.2, 18.01±0.2, 20.47±0.2, 21.02±0.2, 21.35±0.2, 23.17±0.2, 31.05±0.2.

In another preferred embodiment, the X-ray powder diffraction pattern of the crystal form B further has peaks at 2 or more than 2 of diffraction angles 2θ (°) selected from the following group B2: 8.66±0.2, 16.89±0.2, 19.40±0.2, 35.64±0.2.

In another preferred embodiment, the X-ray powder diffraction pattern of the crystal form B further has peaks at 2 or more than 2 of diffraction angles 2θ (°) selected from the following group B3: 13.94±0.2, 16.28±0.2, 17.43±0.2, 20.08±0.2, 20.76±0.2, 22.10±0.2, 22.76±0.2, 24.03±0.2, 24.72±0.2, 25.25±0.2, 26.30±0.2, 26.54±0.2, 28.31±0.2, 28.47±0.2, 28.90±0.2, 32.16±0.2, 36.27±0.2, 39.10±0.2.

In another preferred embodiment, the X-ray powder diffraction pattern of the crystal form B has peaks at 6 or more or all (such as 6, 7, 8, 9, 10, 11, 12, 13, 14, 15, etc.) of diffraction angles 2θ (°) selected from group B1, B2 and B3.

In another preferred embodiment, the X-ray powder diffraction pattern of the crystal form B has peaks at diffraction angles 2θ (°) of table B and the intensity of each peak is shown as in table B:

TABLE B

| 2θ(°) | I/I₀ | 2θ(°) | I/I₀ | 2θ(°) | I/I₀ |
|---|---|---|---|---|---|
| 7.66 | VS | 8.66 | M | 13.57 | S |
| 13.94 | W | 15.36 | S | 16.28 | W |
| 16.89 | M | 17.43 | W | 18.01 | S |
| 19.40 | M | 20.08 | W | 20.47 | S |
| 20.76 | W | 21.02 | S | 21.35 | S |
| 22.10 | W | 22.76 | W | 23.17 | VS |
| 24.03 | W | 24.72 | W | 25.25 | W |
| 26.30 | W | 26.54 | W | 28.31 | W |
| 28.47 | W | 28.90 | W | 31.05 | S |
| 32.16 | W | 35.64 | M | 36.27 | W |
| 39.10 | W | | | | |

In another preferred embodiment, the X-ray powder diffraction pattern of the crystal form B is basically shown as FIG. 4.

In another preferred embodiment, the crystal form B has an exothermic peak at 167.07° C. (as FIG. 5) and an exothermic peak at 254.56° C., and a degradation of 44.78% from 100° C. to 295.08° C. (as FIG. 6).

In another preferred embodiment, the polymorph is C-type crystal of the L-tartrate of a compound of formula X, i.e., crystal form C, the X-ray powder diffraction pattern of which has peaks at diffraction angles 2θ (°) of the following group C1: 8.56±0.2, 11.68±0.2, 13.15±0.2, 15.37±0.2, 15.94±0.2, 16.99±0.2, 19.15±0.2, 22.42±0.2, 25.06±0.2, 25.84±0.2.

In another preferred embodiment, the X-ray powder diffraction pattern of the crystal form C further has peaks at diffraction angles 2θ (°) of the following group C2: 4.33±0.2, 11.08±0.2, 12.22±0.2, 13.87±0.2, 20.62±0.2, 32.44±0.2, 37.06±0.2.

In another preferred embodiment, the X-ray powder diffraction pattern of the crystal form C further has peaks at 2 or more than 2 of diffraction angles 2θ (°) selected from the following group C3: 1.27±0.2, 14.38±0.2, 18.07±0.2, 23.52±0.2, 23.77±0.2, 29.08±0.2.

In another preferred embodiment, the X-ray powder diffraction pattern of the crystal form C has peaks at 6 or more or all (such as 6, 7, 8, etc.) of diffraction angles 2θ (°) selected from group C1, C2 and C3.

In another preferred embodiment, the X-ray powder diffraction pattern of the crystal form C has peaks at diffraction angles 2θ (°) of table C, and the intensity of each peak is shown as in table C:

TABLE C

| 2θ(°) | I/I₀ | 2θ(°) | I/I₀ | 2θ(°) | I/I₀ |
|---|---|---|---|---|---|
| 1.27 | W | 4.33 | M | 8.56 | VS |
| 11.08 | M | 11.68 | VS | 12.22 | M |
| 13.15 | S | 13.87 | M | 14.38 | W |
| 15.37 | S | 15.94 | S | 16.99 | VS |
| 18.07 | W | 19.15 | S | 20.62 | M |
| 22.42 | S | 23.52 | W | 23.77 | W |
| 25.06 | S | 25.84 | S | 29.08 | W |
| 32.44 | M | 37.06 | M | | |

In another preferred embodiment, the X-ray powder diffraction pattern of the crystal form C is basically shown as FIG. 7.

In another preferred embodiment, in the crystal form C, the mole ratio of L-tartaric acid to a compound of formula X is (0.8-2.1):1, preferably (1.0-1.2):1, more preferably 1:1.

In another preferred embodiment, the crystal form C has an exothermic peak at 197.65° C. (as FIG. 8), and a degradation of 4.16% from 100° C. to 177.68° C. and a degradation of 31.36% from 177.68° C. to 295.2° C. (as FIG. 9).

In another preferred embodiment, the polymorph is D-1-type crystal of the phosphate of a compound of formula X, i.e., crystal form D-1, the X-ray powder diffraction pattern of which has peaks at diffraction angles 2θ (°) of the following group D-1-1: 4.30±0.2, 8.55±0.2, 12.79±0.2, 14.20±0.2, 15.61±0.2, 16.60±0.2, 17.17±0.2, 18.04±0.2, 20.74±0.2, 21.46±0.2, 22.36±0.2, 24.79±0.2, 25.51±0.2, 27.04±0.2, 28.72±0.2.

In another preferred embodiment, the X-ray powder diffraction pattern of the crystal form D-1 further has peaks at diffraction angles 2θ (°) of the following group D-1-2:14.86±0.2, 24.23±0.2, 29.71±0.2, 32.20±0.2.

In another preferred embodiment, the X-ray powder diffraction pattern of the crystal form D-1 further has peaks at 2 or more than 2 of diffraction angles 2θ (°) selected from the following group D-1-3:1.20±0.2, 10.00±0.2, 13.39±0.2, 30.92±0.2.

In another preferred embodiment, the X-ray powder diffraction pattern of the crystal form D-1 has peaks at 6 or more or all (such as 6, 7, 8, 9, 10, 11, 12, 13, 14, 15, etc.) of diffraction angles 2θ (°) selected from group D-1-1, D-1-2 and D-1-3.

In another preferred embodiment, the X-ray powder diffraction pattern of the crystal form D-1 has peaks at diffraction angles 2θ (°) of table D-1, and the intensity of each peak is shown as in table D-1:

TABLE D-1

| 2θ(°) | I/I₀ | 2θ(°) | I/I₀ | 2θ(°) | I/I₀ |
|---|---|---|---|---|---|
| 1.20 | W | 4.30 | S | 8.55 | S |
| 10.00 | W | 12.79 | VS | 13.39 | W |
| 14.20 | S | 14.86 | M | 15.61 | VS |
| 16.60 | VS | 17.17 | S | 18.04 | VS |
| 20.74 | VS | 21.46 | VS | 22.36 | S |
| 24.23 | M | 24.79 | S | 25.51 | S |
| 27.04 | S | 28.72 | S | 29.71 | M |
| 30.92 | W | 32.20 | M | | |

In another preferred embodiment, the X-ray powder diffraction pattern of the crystal form D-1 is basically shown as FIG. 10.

In another preferred embodiment, the polymorph is D-2-type crystal of the phosphate of a compound of formula X, i.e., crystal form D-2, the X-ray powder diffraction pattern of which has peaks at diffraction angles 2θ (°) of the following group D-2-1: 4.31±0.2, 12.97±0.2, 14.11±0.2, 14.56±0.2, 15.14±0.2, 16.15±0.2, 17.26±0.2, 20.32±0.2, 21.85±0.2, 24.10±0.2, 25.42±0.2.

In another preferred embodiment, the X-ray powder diffraction pattern of the crystal form D-2 further has peaks at diffraction angles 2θ (°) of the following group D-2-2: 8.66±0.2, 23.14±0.2, 26.99±0.2, 29.62±0.2, 37.81±0.2.

In another preferred embodiment, the X-ray powder diffraction pattern of the crystal form D-2 further has peaks at 2 or more than 2 of diffraction angles 2θ (°) selected from the following group D-2-3:1.08±0.2, 19.59±0.2, 32.07±0.2.

In another preferred embodiment, the X-ray powder diffraction pattern of the crystal form D-2 has peaks at 6 or more or all (such as 6, 7, 8, 9, 10, 11, 12, 13, 14, 15, etc.) of diffraction angles 2θ (°) selected from group D-1-2, D-2-2 and D-2-3.

In another preferred embodiment, the X-ray powder diffraction pattern of the crystal form D-2 has peaks at diffraction angles 2θ (°) of table D-2, and the intensity of each peak is shown as in table D-2:

TABLE D-2

| 2θ(°) | I/I₀ | 2θ(°) | I/I₀ | 2θ(°) | I/I₀ |
|---|---|---|---|---|---|
| 1.08 | W | 4.31 | S | 8.66 | M |
| 12.97 | S | 14.11 | S | 14.56 | S |
| 15.14 | S | 16.15 | VS | 17.26 | VS |
| 19.59 | W | 20.32 | S | 21.85 | VS |
| 23.14 | M | 24.10 | S | 25.42 | S |
| 26.99 | M | 29.62 | M | 32.07 | W |
| 37.81 | M | | | | |

In another preferred embodiment, the X-ray powder diffraction pattern of the crystal form D-2 is basically shown as FIG. 11.

In another preferred embodiment, the polymorph is E-type crystal of the hydrobromide of a compound of formula X, i.e., crystal form E, the X-ray powder diffraction pattern of which has peaks at diffraction angles 2θ (°) of the following group E1: 13.48±0.2, 13.83±0.2, 15.38±0.2, 17.28±0.2, 17.95±0.2, 19.67±0.2, 20.65±0.2, 22.31±0.2, 23.43±0.2, 24.78±0.2, 25.99±0.2, 27.11±0.2, 27.89±0.2, 31.08±0.2, 31.59±0.2.

In another preferred embodiment, the X-ray powder diffraction pattern of the crystal form E further has peaks at diffraction angles 2θ (°) of the following group E2: 8.64±0.2, 20.37±0.2, 21.41±0.2, 21.86±0.2, 23.01±0.2, 23.15±0.2, 25.33±0.2, 32.93±0.2, 33.32±0.2, 33.57±0.2, 33.92±0.2.

In another preferred embodiment, the X-ray powder diffraction pattern of the crystal form E further has peaks at 2 or more than 2 of diffraction angles 2θ (°) selected from the following group E3: 14.43±0.2, 17.64±0.2, 18.77±0.2, 26.52±0.2, 28.99±0.2, 30.79±0.2, 32.13±0.2.

In another preferred embodiment, the X-ray powder diffraction pattern of the crystal form E has peaks at 6 or more or all (such as 6, 7, 8, 9, 10, 11, 12, 13, 14, 15, etc.) of diffraction angles 2θ (°) selected from the following groups E1, E2 and E3.

In another preferred embodiment, the X-ray powder diffraction pattern of the crystal form E has peaks at diffraction angles 2θ (°) of table E, and the intensity of each peak is shown as in table E:

TABLE E

| 2θ(°) | I/I₀ | 2θ(°) | I/I₀ | 2θ(°) | I/I₀ |
|---|---|---|---|---|---|
| 8.64 | M | 13.48 | S | 13.83 | VS |
| 14.43 | W | 15.38 | S | 17.28 | S |
| 17.64 | W | 17.95 | S | 18.77 | W |
| 19.67 | VS | 20.37 | M | 20.65 | VS |
| 21.41 | M | 21.86 | M | 22.31 | VS |
| 23.01 | M | 23.15 | M | 23.43 | S |
| 24.78 | VS | 25.33 | M | 25.99 | VS |
| 26.52 | W | 27.11 | S | 27.89 | S |
| 28.99 | W | 30.79 | W | 31.08 | S |
| 31.59 | S | 32.13 | W | 32.93 | M |
| 33.32 | M | 33.57 | M | 33.92 | M |

In another preferred embodiment, the X-ray powder diffraction pattern of the crystal form E is basically shown as FIG. 12.

In another preferred embodiment, the polymorph is F-type crystal of the fumarate of a compound of formula X, i.e., crystal form F, the X-ray powder diffraction pattern of which has peaks at diffraction angles 2θ (°) of the following group F1: 13.61±0.2, 14.39±0.2, 14.84±0.2, 15.55±0.2, 17.70±0.2, 21.01±0.2, 22.54±0.2, 24.56±0.2, 24.99±0.2.

In another preferred embodiment, the X-ray powder diffraction pattern of the crystal form F has peaks at diffraction angles 2θ (°) of table F, and the intensity of each peak is shown as in table F:

TABLE F

| 2θ(°) | I/I₀ | 2θ(°) | I/I₀ | 2θ(°) | I/I₀ |
|---|---|---|---|---|---|
| 13.61 | S | 14.39 | S | 14.84 | VS |
| 15.55 | VS | 17.70 | S | 21.01 | VS |
| 22.54 | VS | 24.56 | S | 24.99 | S |

In another preferred embodiment, the X-ray powder diffraction pattern of the crystal form F is basically shown as FIG. 14.

In another preferred embodiment, the polymorph is G-1-type crystal of the succinate of a compound of formula X, i.e., crystal form G-1, the X-ray powder diffraction pattern of which has peaks at diffraction angles 2θ (°) of the following group G-1-1:10.78±0.2, 12.94±0.2, 14.47±0.2, 14.98±0.2, 15.31±0.2, 17.59±0.2, 19.63±0.2, 21.82±0.2, 22.57±0.2, 24.25±0.2, 25.29±0.2, 26.02±0.2, 26.65±0.2.

In another preferred embodiment, the X-ray powder diffraction pattern of the crystal form G-1 further has peaks at 2 or more than 2 of diffraction angles 2θ (°) selected from the following group G-1-2: 4.68±0.2, 5.65±0.2, 7.33±0.2, 11.26±0.2, 11.65±0.2, 12.16±0.2, 18.37±0.2, 18.58±0.2, 28.90±0.2.

In another preferred embodiment, the X-ray powder diffraction pattern of the crystal form G-1 further has peaks at 2 or more than 2 of diffraction angles 2θ (°) selected from the following group G-1-3:1.15±0.2, 1.88±0.2, 16.28±0.2, 20.86±0.2, 23.39±0.2, 28.33±0.2, 30.98±0.2, 32.39±0.2.

In another preferred embodiment, the X-ray powder diffraction pattern of the crystal form G-1 has peaks at 6 or more or all (such as 6, 7, 8, 9, 10, 11, 12, 13, 14, etc.) of diffraction angles 2θ (°) selected from group G-1-1, G-1-2 and G-1-3.

In another preferred embodiment, the X-ray powder diffraction pattern of the crystal form G-1 has peaks at diffraction angles 2θ (°) of table G-1, and the intensity of each peak is shown as in table G-1:

TABLE G-1

| 2θ(°) | I/I₀ | 2θ(°) | I/I₀ | 2θ(°) | I/I₀ |
|---|---|---|---|---|---|
| 1.15 | W | 1.88 | W | 4.68 | M |
| 5.65 | M | 7.33 | M | 10.78 | VS |
| 11.26 | M | 11.65 | M | 12.16 | M |
| 12.94 | VS | 14.47 | VS | 14.98 | S |
| 15.31 | S | 16.28 | W | 17.59 | S |
| 18.37 | M | 18.58 | M | 19.63 | VS |
| 20.86 | W | 21.82 | VS | 22.57 | VS |
| 23.39 | W | 24.25 | S | 25.29 | S |
| 26.02 | S | 26.65 | S | 28.33 | W |
| 28.90 | M | 30.98 | W | 32.39 | W |

In another preferred embodiment, the X-ray powder diffraction pattern of the crystal form G-1 is basically shown as FIG. 15.

In another preferred embodiment, the polymorph is G-2-type crystal of the succinate of a compound of formula X, i.e., crystal form G-2, the X-ray powder diffraction pattern of which has peaks at diffraction angles 2θ (°) of the following group G-2-1:10.89±0.2, 11.71±0.2, 13.06±0.2, 14.74±0.2, 15.37±0.2, 17.74±0.2, 18.58±0.2, 19.72±0.2, 20.56±0.2, 21.94±0.2, 22.21±0.2, 22.75±0.2, 24.94±0.2, 26.14±0.2.

In another preferred embodiment, the X-ray powder diffraction pattern of the crystal form G-2 further has peaks at diffraction angles 2θ (°) of the following group G-2-2:12.25±0.2.

In another preferred embodiment, the X-ray powder diffraction pattern of the crystal form G-2 further has peaks at 2 or more than 2 of diffraction angles 2θ (°) selected from the following group G-2-3:1.09±0.2, 5.62±0.2, 7.36±0.2, 8.71±0.2, 11.39±0.2, 16.36±0.2, 23.96±0.2, 24.13±0.2, 29.92±0.2, 31.57±0.2, 33.76±0.2.

In another preferred embodiment, the X-ray powder diffraction pattern of the crystal form G-2 has peaks at 6 or more or all (such as 6, 7, 8, 9, 10, 11, 12, 13, 14, etc.) of diffraction angles 2θ (°) selected from group G-2-1, G-2-2 and G-2-3.

In another preferred embodiment, the X-ray powder diffraction pattern of the crystal form G-2 has peaks at diffraction angles 2θ (°) of table G-2, and the intensity of each peak is shown as in table G-2:

TABLE G-2

| 2θ(°) | I/I₀ | 2θ(°) | I/I₀ | 2θ(°) | I/I₀ |
|---|---|---|---|---|---|
| 1.09 | W | 5.62 | W | 7.36 | W |
| 8.71 | W | 10.89 | S | 11.39 | W |
| 11.71 | S | 12.25 | M | 13.06 | VS |
| 14.74 | VS | 15.37 | S | 16.36 | W |
| 17.74 | VS | 18.58 | S | 19.72 | S |
| 20.56 | S | 21.94 | S | 22.21 | S |
| 22.75 | S | 23.96 | W | 24.13 | W |
| 24.94 | S | 26.14 | S | 29.92 | W |
| 31.57 | W | 33.76 | W | | |

In another preferred embodiment, the X-ray powder diffraction pattern of the crystal form G-2 is basically shown as FIG. 16.

In another preferred embodiment, the polymorph is crystal form I of a compound of formula X, the X-ray powder diffraction pattern of which has peaks at diffraction angles 2θ (°) of the following group I-1: 8.83±0.2, 11.51±0.2, 12.60±0.2, 13.13±0.2, 13.96±0.2, 15.93±0.2, 17.03±0.2, 19.78±0.2, 21.14±0.2, 22.06±0.2, 22.66±0.2, 23.19±0.2, 25.07±0.2.

In another preferred embodiment, the X-ray powder diffraction pattern of the crystal form I further has peaks at 2 or more than 2 of diffraction angles 2θ (°) selected from the following group I-2: 12.46±0.2, 19.46±0.2, 20.45±0.2, 24.10±0.2, 24.70±0.2, 26.81±0.2, 27.27±0.2.

In another preferred embodiment, the X-ray powder diffraction pattern of the crystal form I further has peaks at 2 or more than 2 of diffraction angles 2θ (°) selected from the following group I-3: 17.75±0.2, 19.98±0.2, 26.19±0.2, 26.48±0.2, 27.91±0.2, 28.17±0.2, 28.53±0.2, 30.08±0.2, 30.76±0.2, 31.79±0.2, 32.15±0.2, 34.05±0.2, 36.01±0.2, 37.04±0.2, 37.44±0.2, 38.38±0.2.

In another preferred embodiment, the X-ray powder diffraction pattern of the crystal form I has peaks at 6 or more or all (such as 6, 7, 8, 9, 10, 11, 12, 13, 14, 15, etc.) of diffraction angles 2θ (°) selected from group I-1, 1-2 and 1-3.

In another preferred embodiment, the X-ray powder diffraction pattern of the crystal form I has peaks at diffraction angles 2θ (°) of table I1, and the intensity of each peak is shown as in table I1:

TABLE I1

| 2θ(°) | I/I₀ | 2θ(°) | I/I₀ | 2θ(°) | I/I₀ |
|---|---|---|---|---|---|
| 8.83 | S | 11.51 | S | 12.46 | M |
| 12.60 | VS | 13.13 | S | 13.96 | VS |
| 15.93 | S | 17.03 | VS | 17.75 | W |
| 19.46 | M | 19.78 | VS | 19.98 | W |
| 20.45 | M | 21.14 | S | 22.06 | S |
| 22.66 | VS | 23.19 | S | 24.10 | M |
| 24.70 | M | 25.07 | S | 26.19 | W |
| 26.48 | W | 26.81 | M | 27.27 | M |
| 27.91 | W | 28.17 | W | 28.53 | W |
| 30.08 | W | 30.76 | W | 31.79 | W |
| 32.15 | W | 34.05 | W | 36.01 | W |
| 37.04 | W | 37.44 | W | 38.38 | W |

In another preferred embodiment, the X-ray powder diffraction pattern of the crystal form I is basically shown as FIG. 17.

The crystal form I has a high crystallinity as seen by XRD; the shape of the crystal form I is irregular columnar as seen by a polarizing microscope; there are two exothermic peaks at 177.54° C. and 208.43° C. respectively as shown in FIG. 18; and the DVS curve indicates that the sample almost has no hygroscopicity. The crystal form I has good stability.

In a second aspect of the invention, there is provided a process for preparation of the pharmaceutically acceptable salt of a compound of formula X, or the polymorph of a compound of formula X or a pharmaceutically acceptable salt thereof according to the first aspect of the invention, comprising steps:

(1) in a solvent, compound X1 is deprotected thereby to form a compound of formula X; and

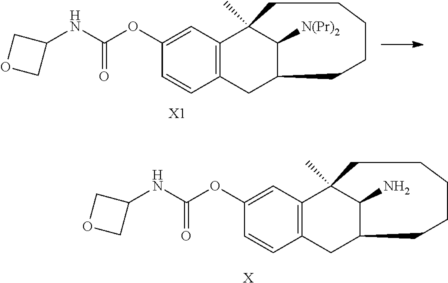

(2) optionally, a compound of formula X and an acid conduct a salt forming reaction thereby to form a pharmaceutically acceptable salt;

(3) optionally, the compound of formula X or its pharmaceutically acceptable salt formed in step (1) or step (2) is subjected to crystallization thereby to obtain a polymorph.

In another preferred embodiment, each of Pr in step (1) is independently hydrogen or a nitrogen protecting group, such as Cbz or Fmoc.

In another preferred embodiment, the method includes any of the following sub-methods:

(A) the polymorph is A-type crystal of the maleate of a compound of formula X, i.e. crystal form A, and step (3) comprises: in a solvent, in the presence of maleic acid, the compound of formula X is subjected to crystallization thereby to form the crystal form A.

In another preferred embodiment, in step (A), the solvent is selected from the group consisting of water, 50% acetone/50% water, acetone, acetonitrile, ethyl acetate, ethanol, isopropanol, 50% acetonitrile/50% water, methanol or tetrahydrofuran, preferably the solvent is acetone, methanol, ethyl acetate or acetonitrile.

In another preferred embodiment, in step (A), the mole ratio of maleic acid to a compound of formula X is (1 to 2):1, preferably (1.0 to 1.2):1.

In another preferred embodiment, in step (A), the crystallization is slowly cooling or addition of an anti-solvent.

In another preferred embodiment, in step (A), the temperature of crystallization is 0 to 80° C.

In another preferred embodiment, in step (A), the time of crystallization is 0.5 hour to 10 days.

(B) the polymorph is B-type crystal of the sulfate of a compound of formula X, i.e. crystal form B, and step (3) comprises: in a solvent, in the presence of sulfuric acid, the compound of formula X is subjected to crystallization thereby to form the crystal form B.

In another preferred embodiment, in step (B), the solvent is selected from the group consisting of water, 50% acetone/50% water, acetone, acetonitrile, ethyl acetate, ethanol, isopropanol, 50% acetonitrile/50% water, methanol or tetrahydrofuran, preferably the solvent is acetone, methanol, ethyl acetate or acetonitrile.

In another preferred embodiment, in step (B), the crystallization is slowly cooling or addition of an anti-solvent.

In another preferred embodiment, in step (B), the temperature of crystallization is 0 to 80° C.

In another preferred embodiment, in step (B), the time of crystallization is 0.5 hour to 10 days.

(C) the polymorph is C-type crystal of the L-tartrate of a compound of formula X, i.e. crystal form C, and step (3) comprises: in a solvent, in the presence of L-tartaric acid, the compound of formula X is subjected to crystallization thereby to form the crystal form C.

In another preferred embodiment, in step (C), the solvent is selected from the group consisting of water, 50% acetone/50% water, acetone, acetonitrile, ethyl acetate, ethanol, isopropanol, 50% acetonitrile/50% water, methanol or tetrahydrofuran, preferably the solvent is acetone, methanol, ethyl acetate or acetonitrile.

In another preferred embodiment, in step (C), the crystallization is slowly cooling or addition of an anti-solvent.

In another preferred embodiment, in step (C), the temperature of crystallization is 0 to 80° C.

In another preferred embodiment, in step (C), the time of crystallization is 0.5 hour to 10 days.

(D-1) the polymorph is D-1-type crystal of the phosphate of a compound of formula X, i.e. crystal form D-1, and step (3) comprises: in a solvent, in the presence of phosphoric acid, the compound of formula X is subjected to crystallization thereby to form the crystal form D-1.

In another preferred embodiment, in step (D-1), the solvent is selected from the group consisting of water, 50% acetone/50% water, acetone, acetonitrile, ethyl acetate, ethanol, isopropanol, 50% acetonitrile/50% water, methanol or tetrahydrofuran, preferably the solvent is acetone, methanol, ethyl acetate or acetonitrile.

In another preferred embodiment, in step (D-1), the crystallization is slowly cooling or addition of an anti-solvent.

In another preferred embodiment, in step (D-1), the temperature of crystallization is 0 to 80° C.

In another preferred embodiment, in step (D-1), the time of crystallization is 0.5 hour to 10 days.

(D-2) the polymorph is D-2-type crystal of the phosphate of a compound of formula X, i.e. crystal form D-2, and step (3) comprises: in a solvent, in the presence of phosphoric acid, the compound of formula X is subjected to crystallization thereby to form the crystal form D-2.

In another preferred embodiment, in step (D-2), the solvent is selected from the group consisting of water, 50% acetone/50% water, acetone, acetonitrile, ethyl acetate, ethanol, isopropanol, 50% acetonitrile/50% water, methanol or tetrahydrofuran, preferably the solvent is acetone, methanol, ethyl acetate or acetonitrile.

In another preferred embodiment, in step (D-2), the crystallization is slowly cooling or addition of an anti-solvent.

In another preferred embodiment, in step (D-2), the temperature of crystallization is 0 to 80° C.

In another preferred embodiment, in step (D-2), the time of crystallization is 0.5 hour to 10 days.

(E) the polymorph is E-type crystal of the hydrobromide of a compound of formula X, i.e. crystal form E, and step (3) comprises: in a solvent, in the presence of hydrobromic acid, the compound of formula X is subjected to crystallization thereby to form the crystal form E.

In another preferred embodiment, in step (E), the solvent is selected from the group consisting of water, 50% acetone/50% water, acetone, acetonitrile, ethyl acetate, ethanol, isopropanol, 50% acetonitrile/50% water, methanol or tetrahydrofuran, preferably the solvent is ethyl acetate.

In another preferred embodiment, in step (E), the crystallization is slowly cooling.

In another preferred embodiment, in step (E), the temperature of crystallization is 0 to 80° C.

In another preferred embodiment, in step (E), the time of crystallization is 0.5 hour to 10 days.

(F) the polymorph is F-type crystal of the fumarate of a compound of formula X, i.e. crystal form F, and step (3) comprises: in a solvent, in the presence of fumaric acid, the compound of formula X is subjected to crystallization thereby to form the crystal form F.

In another preferred embodiment, in step (F), the solvent is selected from the group consisting of water, 50% acetone/50% water, acetone, acetonitrile, ethyl acetate, ethanol, isopropanol, 50% acetonitrile/50% water, methanol or tetrahydrofuran, preferably the solvent is acetonitrile.

In another preferred embodiment, in step (F), the crystallization is addition of an anti-solvent.

In another preferred embodiment, in step (F), the temperature of crystallization is 0 to 80° C.

In another preferred embodiment, in step (F), the time of crystallization is 0.5 hour to 10 days.

(G-1) the polymorph is G-1-type crystal of the succinate of a compound of formula X, i.e. crystal form G-1, and step (3) comprises: in a solvent, in the presence of succinic acid, the compound of formula X is subjected to crystallization thereby to form the crystal form G-1.

In another preferred embodiment, in step (G-1), the solvent is selected from the group consisting of water, 50% acetone/50% water, acetone, acetonitrile, ethyl acetate, ethanol, isopropanol, 50% acetonitrile/50% water, methanol or tetrahydrofuran, preferably the solvent is acetone, methanol or ethyl acetate.

In another preferred embodiment, in step (G-1), the crystallization is slowly cooling or addition of an anti-solvent.

In another preferred embodiment, in step (G-1), the temperature of crystallization is 0 to 80° C.

In another preferred embodiment, in step (G-1), the time of crystallization is 0.5 hour to 10 days.

(G-2) the polymorph is G-2-type crystal of the succinate of a compound of formula X, i.e. crystal form G-2, and step (3) comprises: in a solvent, in the presence of succinic acid, the compound of formula X is subjected to crystallization thereby to form the crystal form G-2.

In another preferred embodiment, in step (G-2), the solvent is selected from the group consisting of water, 50% acetone/50% water, acetone, acetonitrile, ethyl acetate, ethanol, isopropanol, 50% acetonitrile/50% water, methanol or tetrahydrofuran, preferably the solvent is acetonitrile.

In another preferred embodiment, in step (G-2), the crystallization is addition of an anti-solvent.

In another preferred embodiment, in step (G-2), the temperature of crystallization is 0 to 80° C.

In another preferred embodiment, in step (G-2), the time of crystallization is 0.5 hour to 10 days.

(I) the polymorph is crystal form I of a compound of formula X, and step (3) comprises: in a solvent, the compound of formula X is subjected to crystallization thereby to form the crystal form I.

In another preferred embodiment, in the step (I), the solvent is selected from the group consisting of water, methanol, ethanol, propanol, isopropanol, butanol, acetone, acetonitrile, tetrahydrofuran, propylene glycol, ethyl acetate, methyl isobutyl ketone, isopropyl acetate, 2-methyltetrahydrofuran, dichloromethane, methyl tert-butyl ether, dimethyl sulfoxide, toluene, N,N-dimethylacetamide, N-methyl pyrrolidone, or a mixture thereof; preferably is water, methanol, ethanol, isopropanol, acetone, acetonitrile, tetrahydrofuran, ethyl acetate or methyl tert-butyl ether.

In another preferred embodiment, in step (I), the crystallization is slow volatilization or suspended shaking.

In another preferred embodiment, in step (I), 0 to 80° C., preferably 25 to 50° C.

In another preferred embodiment, in step (I), the time of crystallization is 0.5 hour to 10 days.

In a third aspect of the invention, there is provided a pharmaceutical composition comprising:

(a) the pharmaceutically acceptable salt of a compound of formula X or the polymorph of a compound of formula X or a pharmaceutically acceptable salt thereof according to any one of the first aspects of the present invention; and (b) a pharmaceutically acceptable carrier.

In a fourth aspect of the invention, there is provided a use of the pharmaceutically acceptable salt of a compound of formula X or the polymorph of a compound of formula X or a pharmaceutically acceptable salt thereof according to the first aspect of the present invention, or the pharmaceutical composition according to the third aspect of the present invention in the preparation of a drug for the treatment of pain.

In a fifth aspect of the present invention, there is provided a method for treating pain, comprising administering to a subject in need thereof a therapeutically effective amount of the pharmaceutically acceptable salt of a compound of formula X or the polymorph of a compound of formula X or a pharmaceutically acceptable salt thereof according to the first aspect of the present invention or the pharmaceutical composition according to the third aspect of the present invention.

In another preferred embodiment, the pain is acute pain, chronic pain, postoperative pain, pain caused by neuralgia (optionally post-herpetic neuralgia or trigeminal neuralgia), pain caused by diabetic neuropathy, oral pain, pain associated with arthritis or osteoarthritis, or pain associated with cancer or its treatment.

In another preferred embodiment, the pain is neuropathic pain or nociceptive pain.

It is to be understood that within the scope of the present invention, the various technical features of the present invention and the various technical features specifically described hereinafter (as in the embodiments) may be combined with each other to form a new or preferred technical solution. Due to space limitations, we will not repeat them here.

### FIGURES

FIG. 1 is an X-ray powder diffraction pattern of the crystal form A.

FIG. 2 is a differential scanning calorimetry map of the crystal form A.

FIG. 3 is a thermogravimetric analysis pattern of the crystal form A.

FIG. 4 is an X-ray powder diffraction pattern of the crystal form B.

FIG. 5 is a differential scanning calorimetry map of the crystal form B.

FIG. 6 is a thermogravimetric analysis pattern of the crystal form B.

FIG. 7 is an X-ray powder diffraction pattern of the crystal form C.

FIG. 8 is a differential scanning calorimetry map of the crystal form C.

FIG. 9 is a thermogravimetric analysis pattern of the crystal form C.

FIG. 10 is an X-ray powder diffraction pattern of the crystal form D-1.

FIG. 11 is an X-ray powder diffraction pattern of the crystal form D-2.

FIG. 12 is an X-ray powder diffraction pattern of the crystal form E.

FIG. 13 is a differential scanning calorimetry map of the crystal form E.

FIG. 14 is an X-ray powder diffraction pattern of the crystal form F.

FIG. 15 is an X-ray powder diffraction pattern of the crystal form G-1.

FIG. 16 is an X-ray powder diffraction pattern of the crystal form G-2.

FIG. 17 is an X-ray powder diffraction pattern of the crystal form I.

FIG. 18 is a differential scanning calorimetry map of the crystal form I.

FIG. 19 is a thermogravimetric analysis pattern of the crystal form I.

FIG. 20 is a DVS pattern of the crystal form I.

FIG. 21 is a micrograph of the crystal form I.

FIG. 22 is an XRPD pattern of the crystal form A after one week at 60° C.

FIG. 23 is an XRPD pattern of the crystal form A after one week at 40° C./75% RH.

FIG. 24 is an XRPD pattern of the crystal form C after one week at 60° C.

FIG. 25 is an XRPD pattern of the crystal form C after one week at 40° C./75% RH.

### DETAILED DESCRIPTION OF THE INVENTION

After long-term and in-depth research, the inventors unexpectedly discovered a class of benzodicycloalkane derivatives. Compared with the existing dezocine, its potency has been increased more than three-fold, its oral bioavailability has been greatly improved, its drug concentration is constant, its side effects are fewer, which provides better drug selection and better compliance for clinical patients. Studies have also found that a series of polymorphs of the free base of the compound of formula X, its salts and polymorphs of the salts not only have good physical and chemical stability, but also have good pharmacological activities in vivo and in vitro, and thus it has the potential to be further developed into a drug.

### Term

As used herein, “crystal of the present invention”, “crystal form of the present invention”, “polymorph of the present invention” and the like are used interchangeably.

Compound of Formula X

In the present invention, compared with the existing dezocine, the potency of the compound of formula X has been increased more than three-fold, the oral bioavailability of the compound of formula X has been greatly improved, the drug concentration of the compound of formula X is constant, and the side effects of the compound of formula X are fewer.

The present invention also includes a pharmaceutically acceptable salt of a compound of formula X, or a polymorph of a free base of the compound of formula X or a pharmaceutically acceptable salt of the compound of formula X.

In the present invention, the pharmaceutically acceptable salt is selected from the group consisting of hydrochloride, sulfate, hydrobromide, phosphate, methanesulfonate, maleate, L-tartrate, citrate, fumarate, succinate, and besylate.

Polymorph

The solid exist either in an amorphous form or in a crystalline form. In the case of a crystalline form, the molecules are positioned within the three-dimensional lattice. When a compound crystallizes out of a solution or slurry, it can crystallize in different spatial lattices (this property is called “polymorphism”), forming crystals with different crystalline forms, and these various crystalline forms are called “polymorph”. Different polymorphs of a given substance may differ from one another in one or more physical properties such as solubility and dissolution rate, true specific gravity, crystalline form, bulk mode, flowability, and/or solid state stability.

Crystallization

The solubility limit of the compound of interest can be exceeded by operating the solution to complete production-scale crystallization. This can be done in a number of ways, for example by dissolving the compound at relatively high temperatures and then cooling the solution below the saturation limit, or the volume of liquid can be reduced by boiling, atmospheric evaporation, vacuum drying, or by other methods, or the solubility of the compound of interest can be lowered by adding an antisolvent or a solvent in which the compound has a low solubility or a mixture of such a solvent. Another option is to adjust the pH to reduce the solubility. A detailed description of crystallization can be seen in Crystallization, Third Edition, J W Mullens, Butterworth-Heineman Ltd., 1993, ISBN 0750611294.

The term “suspended stirring” as used in the present invention means a method in which the compound of the formula X and the corresponding acid or a solution of the corresponding acid is mixed in a suitable solvent to form a turbid liquid, or the compound of formula X is mixed with a suitable solvent to form a turbid liquid, which is followed by stirring to obtain crystals. The suitable solvent can be water or an organic solvent.

The term “slow volatilization” as used in the present invention refers to a method in which a solution of a compound of the formula X or a solution containing a compound of the formula X and a corresponding acid is slowly volatilized at a certain temperature to obtain a crystal.

The “addition of an anti-solvent” according to the present invention is a method of decomposing a crystal by adding another suitable solvent to a solution of the compound of the formula X.

If salt formation is desired to occur simultaneously with crystallization, if the salt is less soluble than the starting material in the reaction medium, the addition of a suitable acid or base can result in direct crystallization of the desired salt. Similarly, in a medium that the final desired form has less solubility than the reactants, the completion of the synthesis reaction allows the final product to crystallize directly.

Optimization of crystallization can include seeding the crystal in a desired form as a seed in a crystallization solvent. In addition, many crystallization methods use a combination of the above strategies. One embodiment is to dissolve the compound of interest in a solvent at elevated temperatures, followed by controlled addition of an appropriate volume of anti-solvent to bring the system just below the level of saturation. At this point, seed crystals of the desired form (the integrity of the seed crystals are maintained) can be added and the system cooled to complete crystallization.

As used herein, the term “room temperature” generally refers to 4-30° C., preferably 20±5° C.

Polymorph of the Present Invention

The term “polymorph of the present invention” as used herein, includes a polymorph of a compound of formula X, or a pharmaceutically acceptable salt thereof (such as a hydrochloride, a maleate), or a mixture of its various solvates, and also included are different polymorphs of the same salt or solvate.

“Polymorphs of a compound of formula X” and “polymorph of the free base of a compound of formula X” can be used interchangeably. Preferred polymorphs of the present invention include, but are not limited to:

(i) Forms A, B, C, D-1, D-2, E, F, G-1, and G-2 (crystal form of the salt);

(ii) the crystal form I (crystal form of the free base of the compound of formula X).

Identification and Properties of Polymorphs

After preparing the polymorph of the compound of formula X, the present invention has been studied in a variety of ways and instruments as follows.

X-Ray Powder Diffraction

Methods for determining X-ray powder diffraction of crystal forms are known in the art. For example, an X-ray powder diffractometer is used to acquire a spectrum using a copper radiation target at a scanning speed of 2° per minute.

The polymorph of the compound of the formula X of the present invention or a pharmaceutically acceptable salt thereof has a specific crystal form and has a specific characteristic peak in an X-ray powder diffraction (XRPD) pattern.

Differential Scanning Calorimetry

Also known as “differential calorimetric scanning analysis” (DSC), a technique for measuring the relationship between the energy difference between a test substance and a reference material and temperature during heating. The position, shape and number of peaks on the DSC map are related to the nature of the material and can therefore be used qualitatively to identify the substance. This method is commonly used in the art to detect various parameters such as phase transition temperature, glass transition temperature, and reaction heat of a substance.

Pharmaceutical Composition of the Compound of Formula X and Application Thereof

In general, the pharmaceutically acceptable salt of a compound of formula X or the polymorph of a compound of formula X of the present invention can be administered in a suitable dosage form with one or more pharmaceutically acceptable carriers. These dosage forms are suitable for oral, rectal, topical, intraoral, and other parenteral administration (e.g., subcutaneous, intramuscular, intravenous, etc.). For example, dosage forms suitable for oral administration include capsules, tablets, granules, and syrups and the like. The compound of the present invention contained in these preparations may be a solid powder or granule, a solution or suspension in an aqueous or non-aqueous liquid, a water-in-oil or oil-in-water emulsion or the like. The above dosage forms can be prepared from the active compound with one or more carriers or excipients via conventional pharmaceutical methods. The above carriers need to be compatible with the active compound or other excipients. For solid formulations, commonly used non-toxic carriers include, but not limited to, mannitol, lactose, starch, magnesium stearate, cellulose, glucose, sucrose, and the like. Carriers for liquid preparations include water, physiological saline, aqueous dextrose, ethylene glycol, polyethylene glycol, and the like. The active compound can form a solution or suspension with the above carriers.

The compositions of the present invention are formulated, quantified, and administered in a manner consistent with medical practice. The “therapeutically effective amount” of a given compound will be determined by the factors such as the particular condition being treated, the individual being treated, the cause of the condition, the target of the drug, the mode of administration and the like.

The present invention provides that the pharmaceutically acceptable salt of a compound of formula X, or the polymorph of a pharmaceutically acceptable salt of a compound of formula X according to the first aspect of the present invention can be used in the manufacture of a drug for the treatment of pain.

In another preferred embodiment, the pain is acute pain, chronic pain, postoperative pain, pain caused by neuralgia (optionally post-herpetic neuralgia or trigeminal neuralgia), pain caused by diabetic neuropathy, oral pain, pain associated with arthritis or osteoarthritis, or pain associated with cancer or its treatment.

In another preferred embodiment, the pain is neuropathic pain or nociceptive pain.

As used herein, “therapeutically effective amount” refers to an amount that is functional or active to a human and/or animal and that is acceptable to humans and/or animals.

As used herein, “pharmaceutically acceptable carrier” refers to non-toxic, inert, solid, semi-solid substance or a liquid filler, a diluent, an encapsulating material or an auxiliary formulation or any type of excipient that is compatible with the patient which is preferably a mammal and more preferably a human. It is suitable for delivering active agent to a target without terminating the activity of the agent.

As used herein, “patient” refers to an animal, preferably a mammal, and more preferably a human. The term “mammal” refers to a warm-blooded vertebrate mammal, including, for example, cat, dog, rabbit, bear, fox, wolf, monkey, deer, rat, pig and human.

As used herein, “treating” refers to alleviating, delaying, attenuating, preventing, or maintaining an existing disease or disorder (eg, cancer). The treating also includes curing one or more symptoms of the disease or disorder, preventing its development or reducing it to some extent.

The therapeutically effective amount of the pharmaceutical composition or the pharmaceutically acceptable salt of the compound of formula X or the polymorph of the compound of formula X or a pharmaceutically acceptable salt thereof contained in the pharmaceutical composition of the present invention is preferably 0.1 mg-5 g/kg (body weight).

The main advantages of the invention are:

The present inventors have found that polymorphs and salts of the compounds of formula X also have good physicochemical properties and outstanding related pharmacological activities.

### DETAILED DESCRIPTION OF THE INVENTION

The present invention will be further illustrated below with reference to the specific examples. It should be understood that these examples are only to illustrate the invention but not to limit the disclosure of the invention. The experimental methods without specific conditions in the following embodiments are generally carried out according to conventional conditions, or in accordance with the conditions recommended by the manufacturer. Unless indicated otherwise, parts and percentage are calculated by weight.

Reagents and Instruments

In the present invention, the structure and purity of the compound are determined by nuclear magnetic resonance (¹H NMR) and mass spectrometry (LC-MS). ¹H NMR: Bruker AVANCE-400 nuclear magnetic instrument with internal standard tetramethylsilane (TMS). LC-MS: Agilent 1200 HPLC System, 6140 MS Liquid mass spectrometer (purchased from Agilent), column Waters X-Bridge, 150×4.6 mm, 3.5 μm. Preparative High Performance Liquid Chromatography (pre-HPLC): Waters PHW007, column XBridge C18, 4.6*150 mm, 3.5 um.

Use ISCO Combiflash-Rf75 or Rf200 automatic column analyzer, Agela 4 g, 12 g, 20 g, 40 g, 80 g, 120 g disposable silica gel column.

Thin layer chromatography silica gel plate is Yantai Huanghai HSGF254 or Qingdao GF254 silica gel plate, and the silica gel plate used for detecting the reaction by thin layer chromatography (TLC) is 0.15 mm-0.2 mm, and the silica gel plate used for separation and purification by thin layer chromatography is 0.4 mm-0.5 mm. For silica gel, Yantai Huanghai 200-300 mesh silica gel is generally used as a carrier. For basic alumina column, FCP200-300 mesh alkaline alumina for Chinese medicine chromatography is generally used as a carrier.

Unless otherwise stated in the examples, the reactions were all carried out under a nitrogen or argon atmosphere. Unless otherwise stated in the examples, the solution means an aqueous solution.

As used herein, DMF refers to dimethylformamide, DMSO refers to dimethylsulfoxide, THF refers to tetrahydrofuran, DIEA refers to N,N-diisopropylethylamine, EA refers to ethyl acetate, PE refers to petroleum ether, BINAP refers to (2R,3S)-2,2′-bis diphenylphosphino-1,1′-binaphthyl. NBS refers to N-bromosuccinimide, NBS refers to N-chlorosuccinimide, Pd₂(dba)₃ refers to tris(dibenzylideneacetone)dipalladium, Pd(dppf)Cl₂ refers to [1.1′-bis(diphenylphosphino)ferrocene]palladium dichloride,

Acetonitrile ACN, methanol MeOH, ethanol EtOH, isopropanol IPA, acetone ACE, ethyl acetate EA, methyl tert-butyl ether MTBE, tetrahydrofuran THF, water H₂O, 50% acetonitrile 50% ACN.

As used herein, room temperature refers to about 20±5° C.

General Method

X-ray powder diffraction: in the present invention, the powder X-ray diffraction patterns are obtained using a D8 ADVANCE X-ray powder diffraction analyzer through methods known in the art. Test parameters are shown in the following table.

| Parameter | XRPD |
|---|---|
| X-ray | Cu K (λ = 1.54056 Angstrom) |
| tube settings | 40 kV, 40 mA |
| Detector | PSD |
| Scanning | 4°~40° |
| range(°2Theta) | |
| Scanning step | 0.05 |
| (°2Theta) | |
| Scan rate | 1 second/step |

In the pattern, the site of each peak was determined by 2θ(°). It should be understood that different instruments and/or conditions could result in slightly different data and changes in peak site and relative intensity. The division of the intensity of peaks only reflects the approximate size of peaks in each site. In the present invention, the highest diffraction peak of each crystalline form was taken as the base peak which was defined as I₀ with the relative intensity as 100%, (the peak of crystal form I with 2θ(°) value of 13.96 is the base peak, the peak of crystal form A with 2θ(°) value of 23.981 is the base peak, the peak of crystal form B with 2θ(°) value of 23.169 is the base peak, the peak of crystal form C with 2θ(°) value of 16.989 is the base peak, the peak of crystal form D-1 with 2θ(°) value of 21.463 is the base peak, the peak of crystal form D-2 with 2θ(°) value of 21.85 is the base peak, the peak of crystal form E with 2θ(°) value of 22.307 is the base peak, the peak of crystal form F with 2θ(°) value of 21.006 is the base peak, the peak of crystal form G-1 with 2θ(°) value of 10.781 is the base peak, the peak of crystal form G-2 with 2θ(°) value of 14.74 is the base peak), and other peaks had the ratio of their peak height to the peak height of base peak as the relative intensity I/I₀. The definition of the relative intensity of each peak was shown in the following table:

| relative intensity I/I₀(%) | Definition |
|---|---|
| 50~100 | VS (very strong) |
| 25~50 | S (strong) |
| 10~25 | M (medium) |
| 1~10 | W (weak) |

The acid-base molar ratio of the salts of the present invention or their crystalline forms was determined by HPLC/IC or ¹H NMR.

High performance liquid chromatography spectrum was acquired on an Agilent 1260 HPLC.

TGA and DSC pattern were acquired on a TGA Q500 V20.10 Build 36 thermogravimetric analyzer and a DSC Q2000 V24.4 Build 116 differential scanning calorimeter respectively, test parameters are shown in the following table.

| Parameter | TGA | DSC |
|---|---|---|
| Method | Linear warming | Linear warming |
| Sample tray | Platinum plate, open | Aluminum plate, gland |
| Temperature range | 25° C.-set temperature | 25° C.-set temperature |
| Scanning rate | 10 | 10 |
| (° C./min) | | |
| Protective gas | Nitrogen | Nitrogen |

The Dynamic Vapor Sorption (DVS) curve was acquired on the DVS Intrinsic of Surface Measurement Systems. The DVS test parameters are listed in the table below.

| Parameter | Setting value | |
|---|---|---|
| Temperature | 25° | C. |
| Sample size | 10~20 | mg |
| Protective gas | Nitrogen, 0.1M Pa | |
| dm/dt | 0.01%/min | |
| Minimum dm/dt | 5 | min |
| balance time | | |
| The maximum | 120 | min |
| balance time | | |
| RH range | 0% RH~95% RH | |
| RH gradient | 5% | RH |

It should be understood that different values may be obtained when other types of instruments with the same function as the instruments described above or test conditions which are different from the conditions used in the present invention were used. Therefore, the recited value should not be considered as an absolute numerical value.

Due to the instrumental errors or different operators, one skilled in the art will understand that the above parameters used to characterize the physical properties of crystals may differ slightly, so the parameters described above are only used to assist in characterizing the polymorphs provided herein, and can not be regarded as a limitation on the polymorphs of the present invention.

Preparation of Intermediate

Preparation of Intermediate 3a

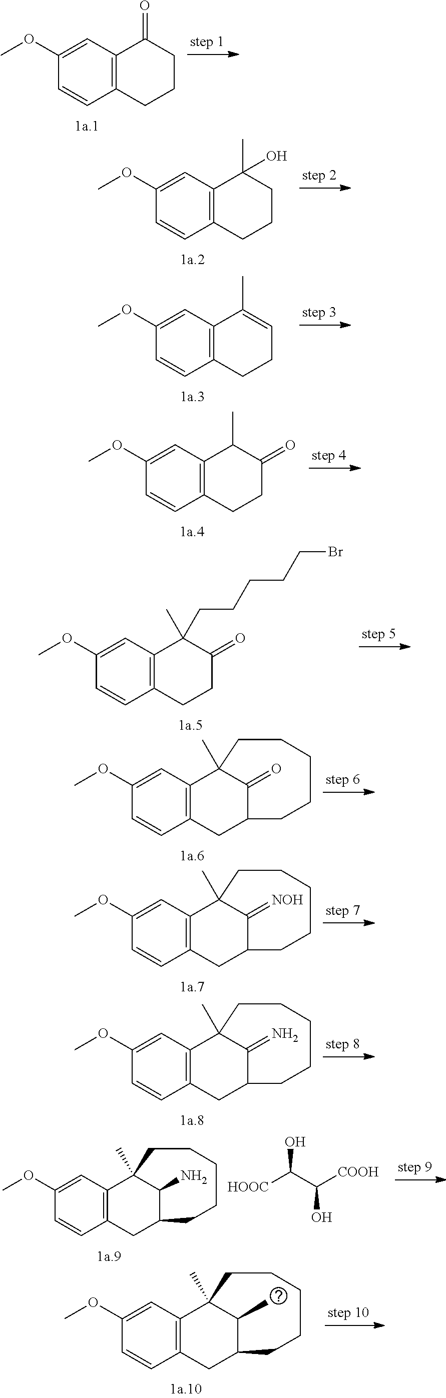

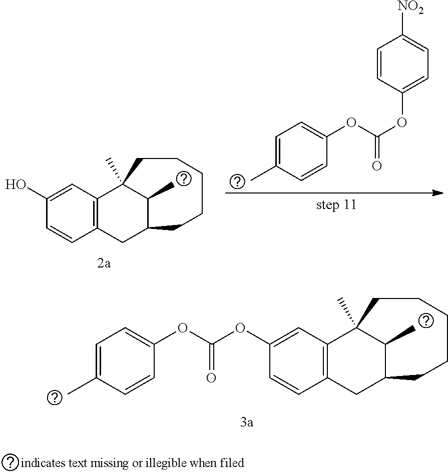 [SMILES: C[C@H]1[C@H]2CCCCC[C@]1(C)c1cc(O)ccc1C2 ; Cc1ccc(OC(=O)Oc2ccc([N+](=O)[O-])cc2)cc1 ; Cc1ccc(OC(=O)Oc2ccc3c(c2)[C@@]2(C)CCCCC[C@@H](C3)[C@@H]2C)cc1]

Step 1: A 2-liter three-necked flask was charged with 300 mL of dry tetrahydrofuran, cooled in an ice bath, and methyl magnesium bromide (350 mL, 3M, 1050 mmol) was placed in a constant pressure titration funnel and dropped into the reaction flask. Compound 1a.1 (60.7 g, 344.73 mmol) was dissolved in 300 mL of dry tetrahydrofuran which was added dropwise at 5-10° C. within 1 hour to the reaction solution. The mixture was stirred at 0-10° C. for 3 hours, and LC-MS was followed till the reaction was completed. The reaction solution was quenched with saturated aqueous ammonium chloride. The organic layer was separated and the aqueous layer was extracted with EA for three times. The organic phases were combined, washed with water and saturated brine, dried over anhydrous Na₂SO₄ and then concentrated under reduced pressure to obtain compound 1a.2 (67 g), which was used directly in the next step. MS m/z (ESI): N/A.

Step 2: Compound 1a.2 (67 g, 348.5 mmol), p-toluenesulfonic acid (59 g, 343 mmol) and 500 mL dry toluene were added to a 1 L three-necked flask, and the mixture was stirred and refluxed for 3 h. The reaction solution was concentrated to remove toluene, and the residue was dissolved in 300 mL of EA, which was washed with saturated sodium bicarbonate solution (100 mL×3) and saturated brine (100 mL). The organic phase was separated, dried over anhydrous Na₂SO₄ and then concentrated under reduced pressure to obtain compound 1a.3 (60 g), which was used directly in the next step. MS m/z (ESI): N/A.

Step 3: A 2 L four-necked flask was charged with 1a.3 (45 g, 258.3 mmol), potassium carbonate (65 g, 470.3 mmol) and dry methylene chloride (500 mL) and the system was cooled under ice-bath. m-CPBA (60 g, 348.3 mmol) was dissolved in 500 mL of dichloromethane, and the solution was added dropwise to the flask within 1.5 h under ice-bath and reacted at 0-10° C. for 1 h. LC-MS was followed till the reaction was completed. The reaction solution was washed with 150 mL of saturated sodium bicarbonate and 100 mL of saturated sodium thiosulfate, and the organic layer was separated, dried over anhydrous Na₂SO₄, and concentrated under reduced pressure to obtain about 400 mL of residue. To the residue was added a solution of trifluoroborane in diethyl ether (360 mg, 49%, 260 mmol). The mixture was warmed to room temperature and stirred for 20 min and then washed with saturated sodium bicarbonate (100 mL×2) and 100 mL of saturated brine. The organic layer was separated, dried and concentrated under reduced pressure to obtain 37.4 g of 1a.4 as a yellow oil. MS m/z (ESI): N/A.

Step 4: A 1 L three-necked flask was charged with 1a.4 (19.4 g, 101.98 mmol), 1,5-dibromopentane (70 g, 304.43 mmol) and 120 mL of toluene and the system was cooled under ice/ethanol-bath. The reaction flask was placed in the dark to avoid light and then TBAB (3.25 g, 10.08 mmol) was added. Then sodium hydroxide solution (35%, 200 g, 1.75 mol) was added dropwise at −2 to 10° C. with stirring, and the mixture was heated to 0-10° C. and stirred for 2 hours, and then reacted at 10-20° C. for 2 hours. LC-MS was followed till the reaction was completed. 100 mL of water was added to the system, which was extracted with toluene (200 mL×3). The organic layer was washed with 2N HCl (100 mL×3) and saturated brine (100 mL×2), dried over anhydrous Na₂SO₄ and then concentrated to remove toluene and evaporated under reduced pressure to remove 1,5-dibromopentane. The residue was purified by preparative liquid chromatography to afford compound 1a.5 (12.8 g, 37%) as a yellow oil. MS m/z (ESI): 356.1[M+NH₄]⁺.

Step 5: A 2 L three-necked flask was charged with 1a.5 (59.2 g, 174.5 mmol) and 600 mL of dry DMF. The mixture was stirred and then sodium hydrogen (15 g, 60%, 375 mmol) was added in batch. The reaction system was slowly warmed to 100° C. and stirred for 1 hour. LC-MS was followed till the reaction was completed. The heating was stopped, and the reaction was cooled under ice-bath and quenched with a saturated ammonium chloride solution, and then 2 L of water was added to the mixed solution. The organic layer was separated and the aqueous layer was extracted with EA (400 mL×3). The organic phases were combined, washed with water (500 mL×2) and saturated brine (500 mL), dried over anhydrous Na₂SO₄ and concentrated. The residue was purified by preparative liquid chromatography to obtain 1a.6 (24 g, 51.1%) as a white solid. MS m/z (ESI): 259.204+Hr.

Step 6: A 500 mL three-necked flask was charged with 1a.6 (5.9 g, 22.84 mmol), hydroxylamine hydrochloride (15.9 g, 228.8 mmol) and 100 mL of pyridine, and the system was stirred at 135° C. overnight. LC-MS was followed till the reaction was completed. The reaction solution was concentrated under reduced pressure to remove pyridine. To the residue was added 100 mL of water, which was extracted with EA (100 mL×2). The organic phases were combined, washed with saturated brine 100 mL, dried over anhydrous Na₂SO₄ and concentrated. The concentrate was beaten with petroleum ether/EA=1:1 and filtered to give 1a.7 (4.93 g, 79%) as a white solid. MS m/z (ESI): 274.2 [M+H]⁺.

Step 7: A 1 L autoclave was charged with 1a.7 (20 g, 73.16 mmol), Raney Ni (23 g), ethanol 400 mL and ammonia water 160 mL (28%-30%), and the system was stirred at 60° C. for 48 hours under 60 atm of hydrogen. The reaction solution was filtered through celite, and the filtrate was concentrated to remove solvent. To the residue was added EA 400 mL and hydrochloric acid/1,4-dioxane (4M, 40 mL). The mixture was stirred at room temperature for 2 hours. The reaction solution was filtered, and the cake was washed with EA and dried to yield 21 g of white solid. The white solid was dissolved in 500 mL of EA and saturated sodium bicarbonate solution (30 mL) was then added dropwise under ice-bath. The organic layer was separated and the aqueous layer was extracted with EA (300 mL). The organic phases were combined, dried and concentrated under reduced pressure to obtain compound 1a.8 (17.4 g, 91.7%) as an oil. MS m/z (ESI): 260.3[M+H]⁺.

Step 8: 1a.8 (41.6 g, 160.38 mmol) was dissolved in 820 mL of methanol and L-tartaric acid (24.1 g, 160.57 mmol) was added and the mixture was stirred at room temperature for 1 hour. To system was added (+)-(L)-seed crystals. The mixture was let stand for two days and filtered. The filtrate was concentrated and saturated sodium bicarbonate solution/EA was added to obtain the free base. The mixture was concentrated again and 20 volume of methanol was added to dissolve the residue, and the same time D-tartaric acid (17.6 g, 117.26 mmol) was added. The mixture was stirred at room temperature for 1 hour. The (−)-(D)-seed crystals were seeded and allowed to stand for 1 day. The mixture was filtered and the cake was recrystallized from methanol to give 1a.9 (17 g, 25.9%) as a white solid. MS m/z (ESI): 410[M+H]⁺.

Step 9: A 250 mL round bottom flask was charged with 1a.9 (5.01 g, 12.235 mmol), Cbz-C1 (2.62 g, 15.307 mmol), potassium carbonate (5.57 g, 40.381 mmol), tetrahydrofuran 50 mL and water 50 mL, and the mixture was stirred at room temperature for 3 hours. LC-MS was followed till the reaction was completed. 200 mL of water was added to the system, which was extracted with EA (100 mL×2). The organic layers were combined, washed with 100 mL of saturated brine, dried over anhydrous Na₂SO₄ and concentrated. The residue was purified by preparative liquid chromatography to obtain compound 1a.10 (5.7 g, 96.8%) as a white solid. MS m/z (ESI): 482.3[M+H]⁺.

Step 10: A 250 mL round bottom flask was charged with 1a.10 (5.7 g, 11.8 mmol) and 80 mL of dry dichloromethane. Under ice-bath boron tribromide (5.9 g, 23.55 mmol) was added, and the mixture was warmed to room temperature and stirred for 3 hours. LC-MS was followed till the reaction was completed. The reaction was quenched by a saturated ammonium chloride solution and the organic layer was separated. The aqueous layer was extracted with methylene chloride. The organic layer was combined and washed with saturated brine, dried over anhydrous Na₂SO₄ and concentrated. The residue was purified by preparative liquid chromatography to obtain compound 2a (3.77 g, 68.2%) as a white solid. MS m/z (ESI): 394.3 [M+H]⁺.

Step 11: A 50 mL three-necked flask was charged with 2a (4 g, 10.54 mmol), bis(p-nitrophenyl) carbonate (3.53 g, 11.6 mmol), DIPEA (2.74 g, 21.2 mmol) and 50 mL of tetrahydrofuran, and the mixture was stirred overnight at room temperature. LC-MS was followed till the reaction was completed. The reaction solution was concentrated to remove the solvent, and the residue was dissolved in 100 mL of EA and washed with 1M sodium hydroxide solution (100 mL×4), 1M hydrochloric acid solution (100 mL×4) and 100 mL of saturated brine, and the organic layer was separated, dried over anhydrous Na₂SO₄ and concentrated to obtain 5.9 g of compound 3a as a yellow solid. MS m/z (ESI): N/A.

### Example 1 Preparing Compound of Formula X

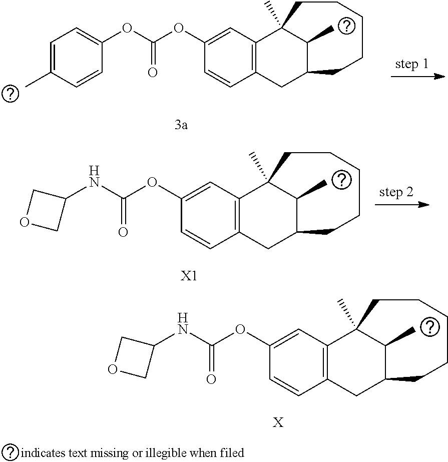

Step 1: a 100 mL round bottom flask was charged with compound 3a (7.45 g, 13.68 mmol), oxetane-3-amine (1 g, 13.68 mmol), tetrahydrofuran 40 mL and DMAP (2.8 g, 23.26 mmol), and the mixture was stirred at room temperature for 1 hour. LC-MS was followed till the reaction was completed. The reaction mixture was concentrated and purified by combi-flash (0-100% EA in Hexane) to obtain compound X1 (5.27 g, 80.6%) as a colorless oil. MS m/z (ESI): 479.3[M+H]⁺.

Step 2: A 500 mL round bottom flask was charged with compound X1 (5.27 g, 11.01 mmol), EA 250 mL and palladium/carbon (5%) 1.05 g, and the mixture was stirred at 50° C. overnight under hydrogen atmosphere. LC-MS was followed until the reaction was completed. The reaction mixture was filtered through celite and concentrated to obtain the free base of compound X (3.5 g, 92.3%) as a white solid. MS m/z (ESI): 345.3[M+H]⁺. ¹H NMR (400 MHz, DMSO-d₆) δ 8.43 (d, J=5.0 Hz, 1H), 7.00 (d, J=8.3 Hz, 1H), 6.88 (s, 1H), 6.77 (d, J=8.3 Hz, 1H), 4.68 (s, 3H), 4.48 (s, 2H), 3.11-2.92 (m, 2H), 2.58 (d, J=16.7 Hz, 1H), 2.17-1.93 (m, 2H), 1.82-1.69 (m, 1H), 1.62-1.33 (m, 7H), 1.23 (s, 3H), 1.17-0.96 (m, 1H), 0.78-0.53 (m, 2H).

### Example 2 Preparing the Crystal Form A of Compound of Formula X

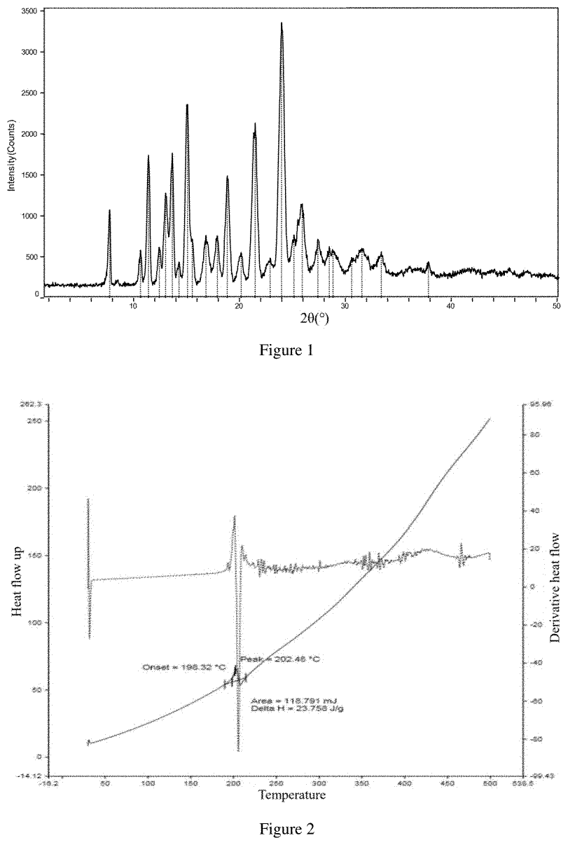

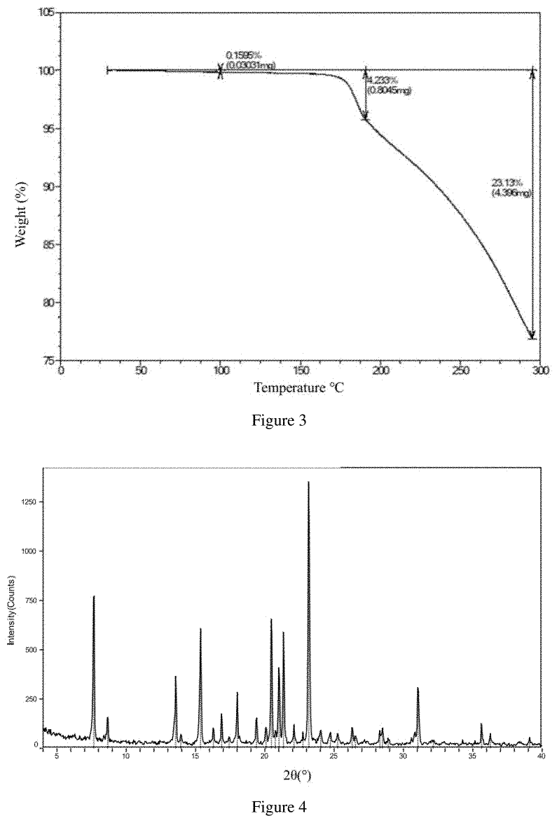

150 mg of the free base of the compound of formula X prepared according to the method of Example 1 was weighed into a glass sample vial, and 525 μL of a 1.0 M maleic acid solution was added at 50° C. After 3 mL of acetonitrile was added, the temperature was kept and the mixture was reaction for 6 h, and the solution was clarified. After 2.6 h, the mixture was slowly cooled down to 0° C., the solution was still clarified. After anti-solvent MTBE was added, an oily solid precipitated, and the solid was obtained by centrifugation. After MTBE was added, a flocculent solid precipitated, and the solid was obtained by centrifugation and dried at 50° C. for 12 h till the solvent was evaporated to give a solid product. The powder X-ray diffraction pattern of the obtained solid product is shown in FIG. 1 (20 angle has been indicated) and the acid-base molar ratio is 1:1, which is defined herein as the crystal form A. Form A has an exothermic peak at 198.32° C. (as FIG. 2), and a degradation of 4.07% from 100° C. to 192.82° C. and a degradation of 18.90% from 192.82° C. to 295.11° C. (as FIG. 3).

### Example 3 Preparing the Crystal Form B of Compound of Formula X

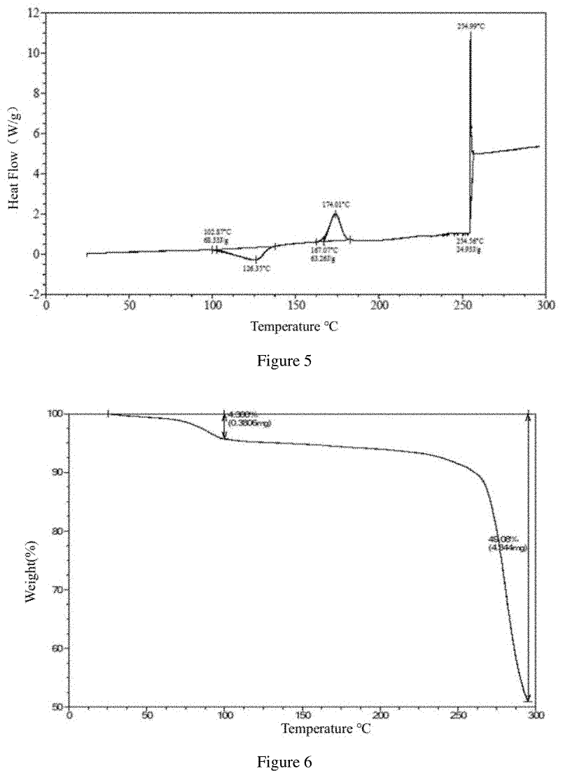

100 mg of the free base of the compound of formula X prepared according to the method of Example 1 was weighed into a glass sample vial, and 695 μL of a 0.5 M sulfuric acid solution was added at 50° C. After 1 mL of methanol was added, the temperature was kept and the mixture was reaction for 6 h, and the solution was clarified. After 2.6 h, the mixture was slowly cooled down to 0° C., and the solution was still clarified. After anti-solvent MTBE was added, a solid precipitated, and the solid was obtained by centrifugation. The solid was dried at 50° C. for 12 h till the solvent was evaporated to give a solid product. The powder X-ray diffraction pattern of the obtained solid product is shown in FIG. 4 (20 angle has been indicated), which is defined herein as the crystal form B. Form B has an exothermic peak at 167.07° C. (as FIG. 5) and an exothermic peak at 254.56° C., and a degradation of 44.78% from 100° C. to 295.08° C. (as FIG. 6).

### Example 4 Preparing the Crystal Form C of Compound of Formula X

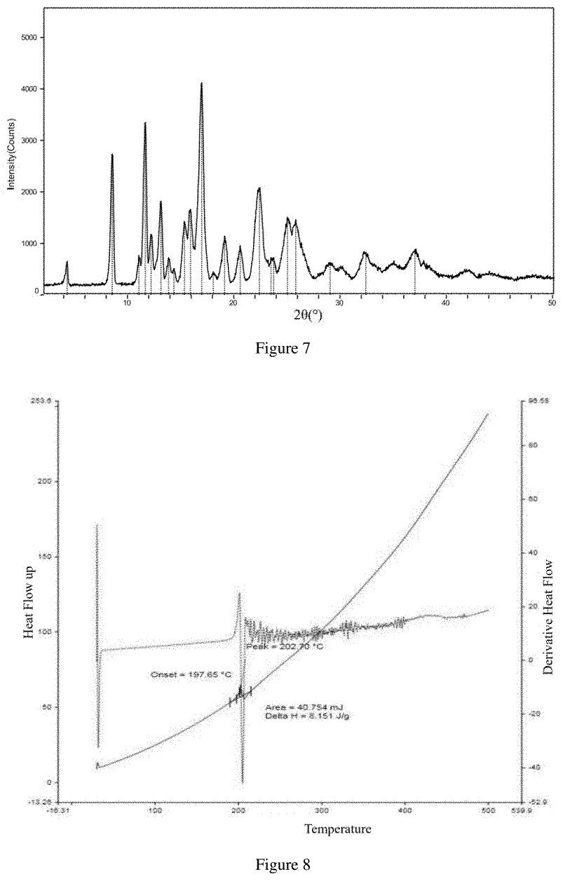

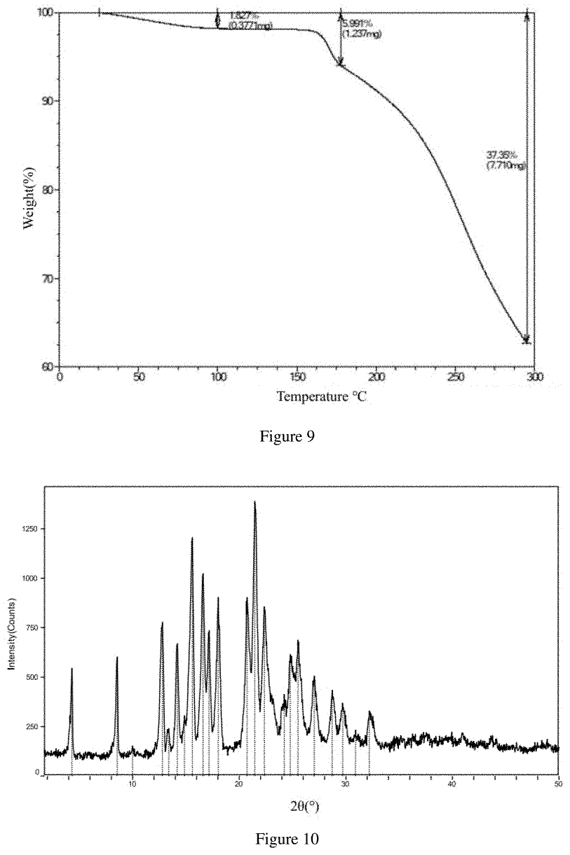

150 mg of the free base of the compound of formula X prepared according to the method of Example 1 was weighed into a glass sample vial, and 525 μL of a 1.0 M L-tartaric acid solution was added at 50° C. After 3 mL of acetone was added, the temperature was kept and the mixture was reaction for 6 h, and the solution was turbid. After 2.6 h, the mixture was slowly cooled down to 0° C., and more solution precipitated and the solid was obtained by centrifugation. The solid was dried at 50° C. for 12 h till the solvent was evaporated to give a solid product. The powder X-ray diffraction pattern of the obtained solid product is shown in FIG. 7 (2θ angle has been indicated) and the acid-base molar ratio is 1:1, which is defined herein as the crystal form C. Form C has an exothermic peak at 197.65° C. (as FIG. 8), and a degradation of 4.16% from 100° C. to 177.68° C. and a degradation of 31.36% from 177.68° C. to 295.2° C. (as FIG. 9).

### Example 5 Preparing Salt of Compound of Formula X or its Crystal Form

20 mg˜40 mg of the free base of the compound of formula X prepared according to the method of Example 1 was weighed, and the corresponding acid was added according to the ratio of acid to free base molar ratio of 1.2:1. Then 1 ml of corresponding solvent was added. The mixture was clarified by heating and ultrasound and reacted at 40° C. for 6 h. The temperature was slowly lowered to precipitate a solid, and the solid was collected by centrifugation. The clarified solution was subjected to an anti-solvent addition method to induce crystallization, and the obtained solid was evaporated to dryness and used for XRPD test. The specific treatment methods and experimental phenomena are shown in Tables 1 and 2.

TABLE 1
the experimental phenomena

| | solvent | | | |
|---|---|---|---|---|
| acid | ethyl acetate | methanol | acetone | acetonitrile |
| hydrochloric | Clarified after | Clarified after | first clarified | first clarified and |
| acid | heating and | heating and stirring; | and then turbid | then turbid by |
| | stirring; | Clarified after | by heating and | heating and |
| | Clarified after | cooling; | stirring; | stirring; |
| | cooling; | Layers was | more solid | more solid |
| | Oily solid | separated and oily | precipitated after | precipitated after |
| | precipitated | solid precipitated | cooling | cooling |
| | after adding | after adding MTBE | more solid | more solid |
| | MTBE | | precipitated after | precipitated after |
| | | | adding MTBE | adding MTBE |
| phosphoric acid | Floccules was | Clarified after | Floccules was | Floccules was |
| | formed after | heating and stirring; | formed after | formed after |
| | heating and | Clarified after | heating and | heating and |
| | stirring; | cooling; | stirring; | stirring; |
| | more solid | solid precipitated | more solid | more solid |
| | precipitated | after adding MTBE | precipitated after | precipitated after |
| | after cooling | | cooling | cooling |
| hydrobromic | first clarified | / | / | / |
| acid | and then turbid | | | |
| | by heating and | | | |
| | stirring; | | | |
| | more solid | | | |
| | precipitated | | | |
| | after cooling | | | |
| methanesulfonic | Clarified after | / | / | Clarified after |
| acid | heating and | | | heating and |
| | stirring; | | | stirring; |
| | Clarified after | | | Clarified after |
| | cooling; | | | cooling; |
| | Solid (colloidal) | | | Solid (colloidal) |
| | precipitated | | | precipitated after |
| | after adding | | | adding MTBE |
| | MTBE | | | |
| citric acid | Clarified after | Clarified after | Clarified after | Clarified after |
| | heating and | heating and stirring; | heating and | heating and |
| | stirring; | Clarified after | stirring; | stirring; |
| | Clarified after | cooling; | Clarified after | Clarified after |
| | cooling; | Solid (oil drop) | cooling; | cooling; |
| | Solid (oil drop) | precipitated after | Solid (oil drop) | Solid (oil drop) |
| | precipitated | adding MTBE | precipitated after | precipitated after |
| | after adding | | adding MTBE | adding MTBE |
| | MTBE | | | |
| fumaric acid | Clarified after | / | / | Clarified after |
| | heating and | | | heating and |
| | stirring; | | | stirring; |
| | Clarified after | | | Clarified after |
| | cooling; | | | cooling; |
| | Layers were | | | Solid (liquid drop) |
| | seperated after | | | precipitated after |
| | adding MTBE | | | adding MTBE |
| succinic acid | Clarified after | NA | Clarified after | Clarified after |
| | heating and | | heating and | heating and |
| | stirring; | | stirring; | stirring; |
| | more solid | | Clarified after | Clarified after |
| | precipitated | | cooling; | cooling; |
| | after cooling | | Solid (oil drop) | Solid (oil drop) |
| | | | precipitated after | precipitated after |
| | | | adding MTBE | adding MTBE |
| benzenesulfonic | Clarified after | Clarified after | Clarified after | Clarified after |
| acid | heating and | heating and stirring; | heating and | heating and |
| | stirring; | Clarified after | stirring; | stirring; |
| | Clarified after | cooling; | Clarified after | Clarified after |
| | cooling; | Solid (oil drop) | cooling; | cooling; |
| | Solid (oil drop) | precipitated after | Solid (oil drop) | Solid (oil drop) |
| | precipitated | adding MTBE | precipitated after | precipitated after |
| | after adding | | adding MTBE | adding MTBE |
| | MTBE | | | |
| sulfuric acid | Floccules was | Clarified after | Clarified after | Clarified after |
| | formed after | heating and stirring; | heating and | heating and |
| | heating and | Clarified after | stirring; | stirring; |
| | stirring; | cooling; | Clarified after | Clarified after |
| | more solid | solid precipitated | cooling; | cooling; |
| | precipitated | after adding MTBE | Layers was | Layers was |
| | after cooling | | separated and | separated and oily |
| | | | oily solid | solid precipitated |
| | | | precipitated after | after adding MTBE |
| | | | adding MTBE | |
| maleic acid | Floccules was | Clarified after | Clarified after | Clarified after |
| | formed after | heating and stirring; | heating and | heating and |
| | heating and | Clarified after | stirring; | stirring; |
| | stirring; | cooling; | Clarified after | Clarified after |
| | more solid | solid precipitated | cooling; | cooling; |
| | precipitated | after adding MTBE | solid | solid precipitated |
| | after cooling | | precipitated after | after adding MTBE |
| | | | adding MTBE | |
| L-tartaric acid | turbid after | Clarified after | turbid after | turbid after heating |
| | heating and | heating and stirring; | heating and | and stirring; |
| | stirring; | Clarified after | stirring; | more solid |
| | more solid | cooling; | more solid | precipitated after |
| | precipitated | solid precipitated | precipitated after | cooling |
| | after cooling | after adding MTBE | cooling | |

Note
1: N/A means solids are not obtained;
2: / means there is no test data.

TABLE 2
treatment

| | solvent | | | |
|---|---|---|---|---|
| acid | ethyl acetate | methanol | acetone | acetonitrile |
| hydrochloric | slowly | N/A | slowly | slowly |
| acid | cooling, | | cooling, | cooling, |
| | adding | | adding | adding |
| | anti-solvent | | anti-solvent | anti-solvent |
| sulfuric acid | slowly | adding | adding | adding |
| | cooling, | anti-solvent | anti-solvent | anti-solvent |
| phosphoric acid | slowly | adding | slowly | slowly |
| | cooling, | anti-solvent | cooling, | cooling, |
| maleic acid | slowly | adding | adding | adding |
| | cooling, | anti-solvent | anti-solvent | anti-solvent |
| hydrobromic | slowly | / | / | / |
| acid | cooling, | | | |
| methanesulfonic | adding | / | / | adding |
| acid | anti-solvent | | | anti-solvent |
| L-tartaric acid | slowly | adding | slowly | slowly |
| | cooling, | anti-solvent | cooling, | cooling, |
| citric acid | adding | adding | adding | adding |
| | anti-solvent | anti-solvent | anti-solvent | anti-solvent |
| fumaric acid | N/A | / | / | adding |
| | | | | anti-solvent |
| succinic acid | slowly | N/A | adding | adding |
| | cooling, | | anti-solvent | anti-solvent |
| benzenesulfonic | adding | adding | adding | adding |
| | anti-solvent | anti-solvent | anti-solvent | anti-solvent |

Note
1: N/A means solids are not obtained;
2: / means there is no test data.

TABLE 3
summary of salting

| | solvent | | | |
|---|---|---|---|---|
| acid | ethyl acetate | methanol | acetone | acetonitrile |
| hydrochloric | amorphous | N/A | amorphous | amorphous |
| acid | form | | form | form |
| sulfuric acid | amorphous | Form B | amorphous | amorphous |
| | form | | form | form |
| phosphoric acid | Form D-1 | Form D-2 | Form D-1 | Form D-1 |
| maleic acid | Form A | amorphous | Form A | Form A |
| | | form | | |
| hydrobromic | Form E | / | / | / |
| acid | | | | |
| methanesulfonic | amorphous | / | / | amorphous |
| acid | form | | | form |
| L-tartaric acid | Form C | Form C | Form C | Form C |
| citric acid | amorphous | amorphous | amorphous | amorphous |
| | form | form | form | form |
| fumaric acid | N/A | / | / | Form F |
| succinic acid | Form G-1 | Form G-1 | Form G-1 | Form G-2 |
| benzenesulfonic | amorphous | amorphous | amorphous | amorphous |
| acid | form | form | form | form |

Note
1: N/A means solids are not obtained;
2: / means there is no test data.

### Example 6 Preparing the Crystal Form I of the Free Base of Compound of Formula X

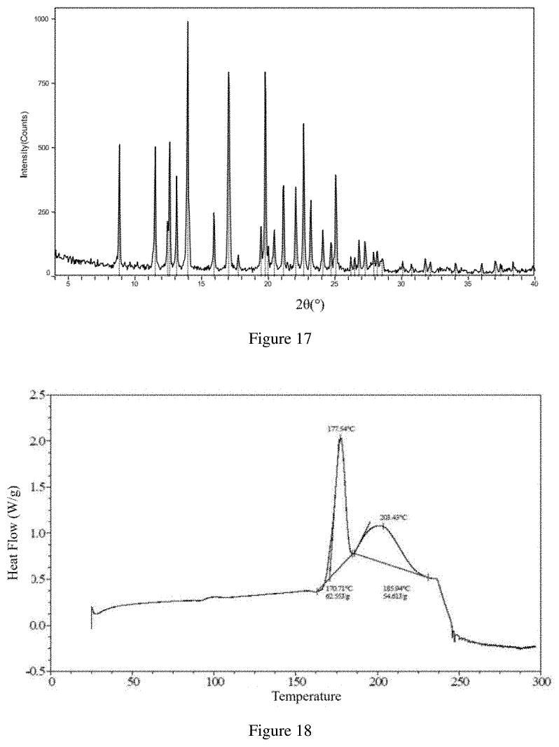

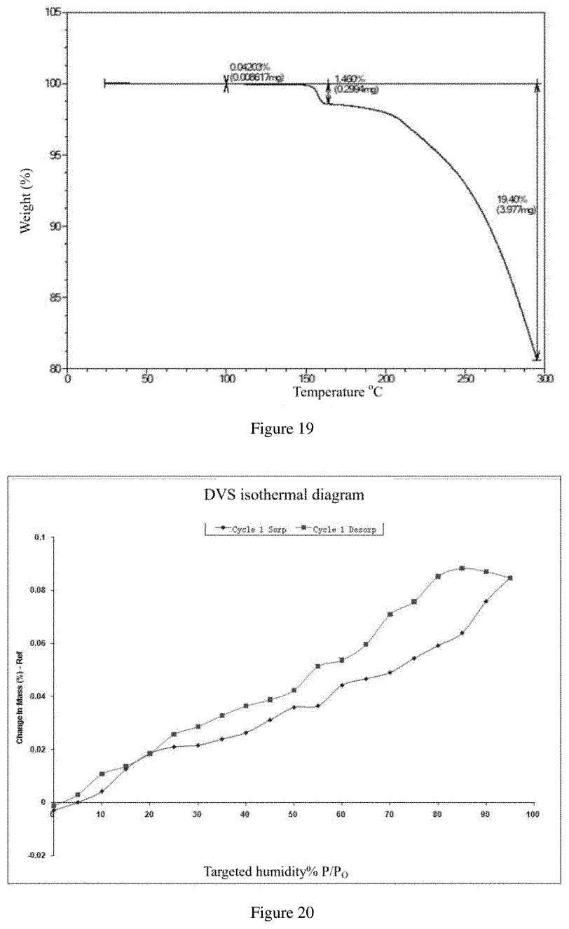

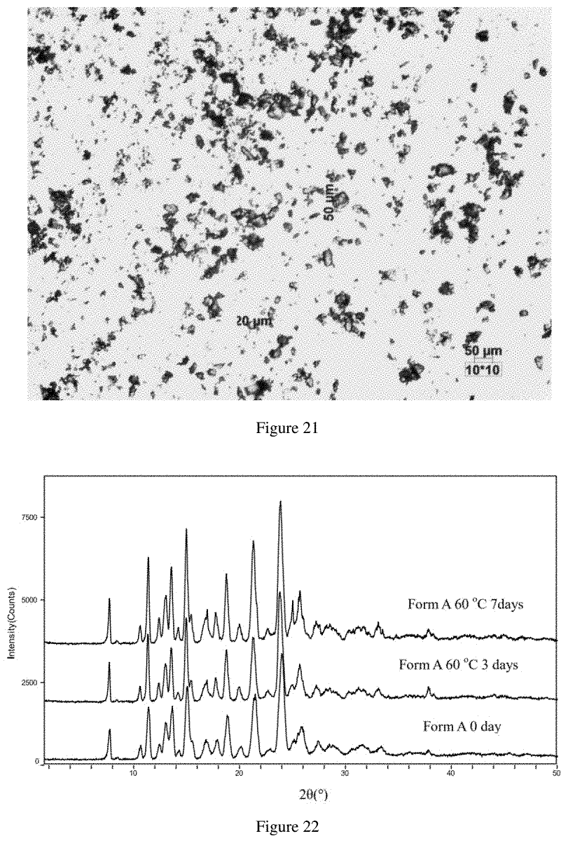

About 10 mg of the free base of the compound of formula X prepared according to the method of Example 1 was weighed into a glass vial, and an appropriate amount of ethyl acetate was added to obtain a nearly saturated solution, which was fully dissolved by ultrasonication and filtered. 20-200 uL of corresponding solvent was added to the clear filtrate, which was stood and the solvent was slowly evaporated at room temperature. After the solvent was completely evaporated, the resulting solid was collected and subjected to an XRPD test. The resulting powder X-ray diffraction pattern is shown in FIG. 17 (2 theta angle has been indicated) and is defined herein as the crystal form I of the free base of the compound of formula X. The crystal form I has a high crystallinity as seen from XRD; the shape of the crystal form I is irregular columnar as seen by a polarizing microscope; there are two exothermic peaks at 177.54° C. and 208.43° C. respectively as shown in FIG. 18; and the DVS curve indicates that the sample almost has no hygroscopicity. The crystal form I has good stability.

### Example 7 Preparing the Crystal Form I of the Free Base of Compound of Formula X

Nine 25° C. suspension shaking tests were set up using different solvent systems. About 20 mg of the free base of the compound of formula X prepared in the same manner as in Example 1 was weighed into a glass vial, and 1 mL of the organic reagent selected in Table 4 was added (in which regarding to THF, ACE, ACN, MeOH, and EA, 20 mg of sample was weighed and 0.2 mL of the organic reagent was added dropwise). The vial was tightly capped and sealed with a sealing film to prevent liquid volatilization, and shaken at 50° C., 225 r/min for one day. Then the vial was taken out. The mixture was centrifuged at 4° C., 14000/min for 15 min, and the supernatant was decanted, and the solid was stood at room temperature and slowly evaporated overnight. The obtained solid was collected and subjected to an XRPD test.

TABLE 4
mixing shaken test at 50° C.

| | form of solid after | | form of solid after |
|---|---|---|---|
| | being shaked for | | being shaked |
| solvent | 1 day | solvent | for 1 day |
| MeOH | Totally dissolved | EtOH | Form I |
| ACN | Form I | EtOAc | Form I |
| MTBE | Form I | H₂O | Form I |
| IPA | Form I | THF | Form I |
| ACE | Totally dissolved | | |

### Example 8 Preparing the Crystal Form I of the Free Base of Compound of Formula X

Nine 25° C. suspension shaking tests were set up using different solvent systems. About 15 mg of the free base of the compound of formula X prepared in the same manner as in Example 1 was weighed into a glass vial, and 1 mL of the organic reagent selected in Table 5 was added (in which regarding to THF, ACE, ACN, MeOH, and EA, 30 mg of sample was weighed and 0.4 mL of the organic reagent was added dropwise). The vial was tightly capped and sealed with a sealing film to prevent liquid volatilization, and shaken at 25° C., 25 r/min. Then the vial was taken out. The mixture was centrifuged at 4° C., 14000/min for 15 min, and the supernatant was decanted, and the solid was stood at room temperature and slowly evaporated overnight. The obtained solid was collected and subjected to an XRPD test. The assay result was show in table 5:

TABLE 5
mixing shaken test at 25° C.

| | form of solid after | form of solid after being |
|---|---|---|
| solvent | being shaked for 1 day | shaked for 7 day |
| MeOH | Form I | dissolved |
| EtOH | Form I | amorphous form |
| ACN | Form I | Form I |
| EtOAc | Form I | dissolved |
| MTBE | Form I | Form I |
| ACE | dissolved | dissolved |
| IPA | Form I | Form I |
| H₂O | Form I | Form I |
| THF | dissolved | Form I |

### Example 9 Stability Test

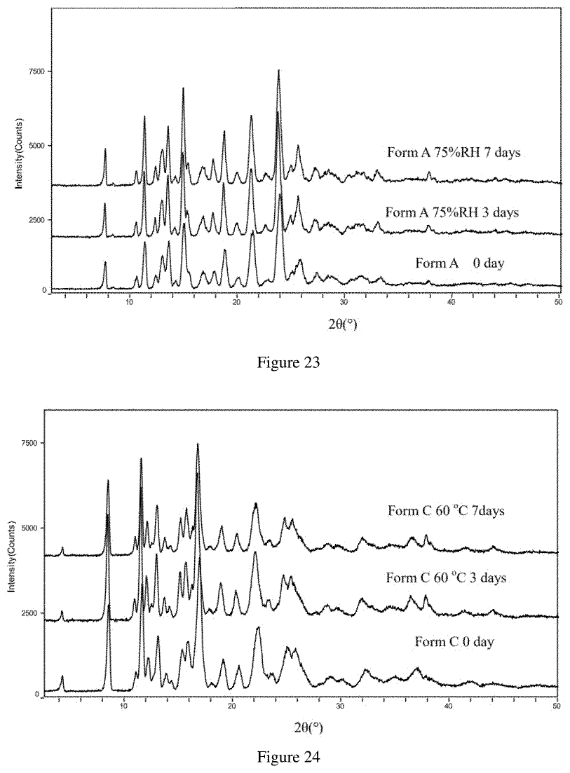

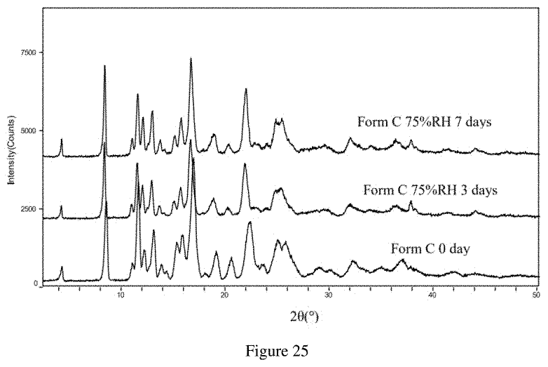

An appropriate amount of the sample was weighed at 60° C. and stored at 40° C., 70% RH, and at the same time another set of sample was sealed and stored at 5° C. as a control. The crystal form and purity change were measured respectively on 3rd and 7th day. The results are shown in Table 6, FIGS. 22, 23, 24 and 25. The two salt types have new impurities, the total impurities are stable, and the crystal form is not changed under the conditions of 60° C. and 40° C., 70% RH.

TABLE 6
results of content and related substance

| | | | | | | | | | | | Total | | |
|---|---|---|---|---|---|---|---|---|---|---|---|---|---|
| | | RRT(%) | | | | | | | | | impurity | Area | |
| Sample | condition | 0.70 | 0.76 | 0.81 | 1.14 | 1.25 | 1.29 | 1.53 | 1.59 | 1.75 | (%) | (%) | character |
| Free base | 5° C., 0 d | — | 1.19 | — | — | — | 3.35 | 0.18 | 0.97 | 0.43 | 6.12 | 93.88 | off-white solid |
| From A | | — | | | | 0.18 | | | | | 0.18 | 99.82 | off-white solid |
| From C | | — | | | | | | | | | | 100.00 | off-white solid |
| From A | 60° C., 3 d | | | 0.12 | | 0.20 | | | | | 0.32 | 99.68 | off-white solid |
| From C | | 3.68 | | 0.24 | 1.44 | | | | | | 5.36 | 94.64 | off-white solid |
| From A | 60° C., 7 d | | | 0.11 | | 0.30 | | | | | 0.42 | 99.58 | off-white solid |
| From C | | 3.60 | | 0.26 | 1.52 | | | | | | 5.38 | 94.62 | off-white solid |
| From A | 40° C./75% RH, 3 d | | | 0.39 | | 0.14 | | | | | 0.53 | 99.47 | off-white solid |
| From C | | 3.60 | | 0.54 | 0.93 | | | | | | 5.08 | 94.92 | off-white solid |
| From A | 40° C./75% RH, 7 d | | | 0.54 | | 0.17 | | | | | 0.72 | 99.28 | off-white solid |
| From C | | 3.04 | | 0.85 | 0.92 | | | | | | 4.81 | 95.19 | off-white solid |

—: below detection limit;
NA: not detected

### Example 10 Solubility Test

The solubility of the crystal form A, the crystal form C and the free base in 0.1 M HCL, pH 4.5, or pH 6.8 buffer or water was tested at room temperature. In the test, the standard curves of the crystal form A, the crystal form C and the free base were plotted respectively. Subsequently, a appropriate amount of the crystal form A, the crystal form C and the free base was weighed respectively, to which was added a appropriate amount of solvent. The mixture was shaken at room temperature for 24 h, centrifuged. The supernatant was taken and the solubility was determined. The results are shown in Table 7, (the concentration unit is mg/mi). Compared to the free base, the solubility of the two salt forms in pH 4.5, pH 6.8, or 0.1 M HCL was significantly improved, in which the crystal form C was increased significantly.

TABLE 7
Test results of solubility of salts and free base at room temperature (unit: mg/ml)

| | solvent of [symbol] | | | | | | | | | | | |
|---|---|---|---|---|---|---|---|---|---|---|---|---|
| | pH 4.5 | | | pH 6.8 | | | 0.1M HCL | | | H₂O | | |
| Form | | phenom- | Final | | phenom- | Final | | phenom- | Final | | phenom- | Final |
| solid | solubility | enon | pH | solubility | enon | pH | solubility | enon | pH | solubility | enon | pH |
| Free base | 3.75 | turbid | 6.41 | 2.03 | turbid | 6.78 | 18.86 | Flocculent | 1.30 | 0.05 | turbid | 8.61 |
| | | | | | | | | precipitate | | | | |
| Form A | ≥8.36 | clarified | 3.92 | ≥11.03 | clarified | 5.86 | 17.84 | Flocculent | 0.91 | 8.25 | Flocculent | 3.86 |
| | | | | | | | | precipitate | | | precipitate | |
| Form C | ≥25.50 | clarified | 3.47 | ≥26.11 | clarified | 3.84 | ≥33.87 | clarified | 1.53 | ≥26.05 | clarified | 3.45 |

[symbol]

indicates data missing or illegible when filed

### Example 11: In Vivo Test of Rats

LC-MS/MS method was applied for the determination of the drug concentration in plasma at different times after the example compounds were orally administered to rats in order to study the pharmacokinetic behavior of the compounds of the invention in vivo in rats and evaluate their pharmacokinetic characteristics.

Protocol:

Test Animals: healthy Adult male SD rats (weight 200-300 g, 3, fasted), provided by SLAC company;

Administration and Dosage: (16 mg/kg or 24 mg/kg, 10 mL/kg, 5% 1,2-propanediol (1,2-Propanediol, Shanghai Titan Technology Co., Ltd. Lot No.: P1057349)) was administered via oral gavage to SD rats;

Blood collection: firstly, the animals which were selected to meet the test requirements prior to administration were weighed. The rats were bound before the blood collection, blood from each administered rat was taken at predetermined time points, (blood was collected at 0.083, 0.25, 0.5, 1, 2, 4, 6, 8 h before and after administration respectively, 9 time points in total), about 150 μl of blood was collected via orbital vein. Blood was transferred to a 1.5 ml tube to which K2EDTA was added previously. The collected blood sample was put on ice, and centrifuged to obtain plasma sample (2000 g, 5 min under 4° C.) within 15 minutes. All the plasma samples were stored at approximately −70° C. until analysis.

LC/MS/MS method was applied to determine the concentrations of the drug. At the same dose and administration, pharmacokinetic parameters of dezocine and some example compounds of the invention in rats were shown in Table 8 and Table 9, Frei is the relative bioavailability, the formula is: (AUC_{compound}/AUC_{dezocine})*100. mpk represents mg/kg.

TABLE 8
Pharmacokinetic parameter after oral administration
of dezocine in rats (the dose of dezocine is 10mpk)

| Compound | AUC(hr*ng/mL) |
|---|---|
| dezocine | 58.6 |

TABLE 9
Pharmacokinetic parameters of dezocine after oral administration
of test compounds in rats (the dose of dezocine is 10mpk)

| Compound No. | AUC(hr*ng/mL) | Fᵣₑₗ(%) |
|---|---|---|
| free base of Compound X | 104 | 177 |

By comparison between Table 8 and Table 9, it can be seen that the compounds of formula X of the present invention have better pharmacokinetic properties as compared with the oral administration of dezocine, the relative bioavailability is greatly improved, the drug effect time is prolonged by more than 2 times, and the dosage and dosing frequency are reduced, the caused side effects are fewer.

### Example 12 Inhibition of hERG Potassium Ion Channel

2.1 Cell Culture

2.1.1 Cells used in this experiment are CHO cell lines (supplied by Sophion Bioscience, Denmark) which are hERG cDNA transfectant and stably express hERG channels, cell progeny is P15. Cells are cultured in medium containing the following ingredients Invitrogen): Ham's F12 medium, 10% (v/v) inactivated fetal bovine serum, 100 μl/ml hygromycin B, 100 μl/ml Geneticin.

2.1.2 CHO hERG cells were grown in Petri dishes containing the above medium and cultured in an incubator containing 5% CO2 at 37° C. CHO hERG cells were transferred onto round glass plates in Petri dishes, and grown on the same culture medium and culture conditions as above 24 h to 48 h prior to the electrophysiological experiments, and the density of CHO hERG cells on each round glass plate needs to meet the requirements that the vast majority of cells are independent and individual.

2.2 Experimental solution The following solutions (recommended by Sophion) were used for electrophysiological recording. The reagents used in this test were provided by Sigma.

TABLE 10
Intracellular and extracellular fluid composition

| | Extracellular | Intracellular fluid |
|---|---|---|
| Reagent | fluid (mM) | (mM) |
| CaCl₂ | 2 | 5.37 |
| MgCl₂ | 1 | 1.75 |
| KCl | 4 | 120 |
| NaCl | 145 | — |
| Glucose | 10 | — |
| HEPES | 10 | 10 |
| EGTA | — | 5 |
| Na-ATP | — | 4 |
| PH | 7.4 | 7.25 |
| | (adjusted with NaOH) | (adjusted with KOH) |
| Osmotic | Osmotic pressure~305 | Osmotic pressure~295 |
| pressure | mOsm | mOsm |

2.3 Electrophysiological Recording System

In this experiment, whole-cell current recording was performed using a manual patch clamp system (HEKA EPC-10 signal amplification and digital conversion system, purchased from HEKA Electronic, Germany) The round glass slide of which surface CHO hERG cells were grown on was placed in an electrophysiological recording slot under an inverted microscope. Perfused steadily with extracellular fluid in recording slot (approximately 1 ml per minute). A conventional whole-cell patch clamp current recording technique was used in the experiment. Unless otherwise specified, experiments were performed at normal room temperature (˜25° C.). Cell clamping was at −80 mV. Cell clamping voltage depolarized to +20 mV to activate hERG potassium channel, clamping to −50 mV after 5 sec to eliminate inactivation and generate tail currents. The tail current peak was used as a value for hERG current. The hERG potassium current recorded in the above steps should be superfused for test drug after the steady perfusion state of the extracellular fluid in the recording slot is stabilized until the inhibition of the hERG current by the drug reached a steady state. The last coincidence of the three consecutive current recording lines was generally used as a criterion to determine whether the state is stable. After reaching a steady state, perfused with extracellular fluid until hERG current returned to the value before the drug adding. One or more drugs could be tested on a single cell, or multiple concentrations of the same drug, but needed to be rinsed with extracellular fluid between different drugs. Cisapride (purchased from Sigma) was used as a positive control in experiments to ensure that the quality of the used cells were normal.

2.4 Compound Treatment and Dilution

The compound was first dissolved in DMSO to a concentration of 10 mM and then the compound was diluted 1000-fold to the final 10 μM test concentration using an extracellular solution. The final concentration of DMSO in the compound test solution was equal to 0.1%. The test concentration of positive control cisapride was 0.1 μM. All stock solutions and test solutions were subjected to regular 5-10 minute sonication and shaking to ensure complete dissolution of the compound.

2.5 Data Analysis

The test data were analyzed by the data analysis software provided by HEKA Patchmaster (V2x73.2), Microsoft Excel and Graphpad Prism 5.0.

Example 11 Inhibition of hERG potassium ion channel

| Compound No. | hERG Inhibition/10 μM |
|---|---|
| free base of Compound X | 20.59% |

It can be seen from Table 11 that compounds of formula X of the present invention have little inhibitory activity on the hERG potassium ion channel and thus have a selective inhibitory effect on the potassium ion channel.

### Example 13 Inhibition of CYP Enzyme

1. Preheat 0.1 M potassium phosphate buffer (K-buffer), pH 7.4:

100 mM K-Buffer: mix 9.5 mL Stock A into 40.5 mL Stock B, bring total volume to 500 mL with Milli-Q water, titrate the buffer with KOH or H3PO4 to pH 7.4.

Stock A (1 M monobasic potassium phosphate): 136.5 g of monobasic potassium phosphate in 1 L of Milli-Q water.

Stock B (1 M dibasic potassium phosphate): 174.2 g of dibasic potassium phosphate in 1 L of Milli-Q water.

2. Prepare serial dilution for test compounds and reference inhibitors (400×) in a 96-well plate:

2.1 Transfer 8 μL of 10 mM test compounds to 12 μL of ACN.

2.2 Prepare individual inhibitor spiking solution for reference: 8 μL of DMSO stock to 12 μL of ACN.

2.3 Prepare 1:3 serial dilutions in DMSO:ACN mixture (v/v: 40:60).

3. Prepare 4×NADPH cofactor (66.7 mg NADPH in 10 mL 0.1 M K-buffer, pH7.4).

4. Prepare 4×substrate (2 mL for each isoform) as indicated in the table below (add HLM where required on ice).

5. Prepare 0.2 mg/mL HLM solution (10 μL of 20 mg/mL to 990 μL of 0.1 M K-buffer) on ice.

6. Add 200 μL of 0.2 mg/mL HLM to the assay wells and then add 1 μL of test compounds or reference compounds into the designated wells on ice.

7. Add following solutions (in duplicate) in a 96-well assay plate on ice:

7.1 Add 30 μL of 2×test compounds and reference compound in 0.2 mg/mL HLM solution;

7.2 Add 15 μL of 4×substrate solution.

8. Pre-incubate the 96-well assay plate and NADPH solution at 37° C. for 5 minutes.

9. Add 15 μL of pre-warmed 8 mM NADPH solution to into the assay plates to initiate the reaction.

10. Incubate the assay plate at 37° C. 5 min for 3A4.

11. Stop the reaction by adding 120 μL of ACN containing IS.

12. After quenching, shake the plates at the vibrator (IKA, MTS 2/4) for 5 min (600 rpm/min) and then centrifuge at 3750 rpm for 15 min (Allegra X-12R centrifuge).

13. Transfer 50 μL of the supernatant from each well into a 96-well sample plate containing 70 μL of wastons water for LC/MS analysis.

TABLE 12
System for CYP3A4 inhibition assay

| | | | | | | | | |
|---|---|---|---|---|---|---|---|---|
| | | | | | | | Final | Microsomes |
| | Probe | Stock | Final | | Selective | Stock | highest | Conc. |
| CYP | Substrate | Conc. | Conc. | CYP | Inhibitors | Conc. | Conc. | (mg/mL) |

| | | | | | | |
|---|---|---|---|---|---|---|
| 3A4 | Midazolam | 3A4 | Ketoconazole | 2.5 mM in | 2.5 | 0.1 |
| | (abbreviated as M) | | | DMSO | | |
| | 1 mM in ACN 5 | | | | | |

TABLE 13
Preparation methods for three types of subtype 3A4

| CYP | 2 mL | 1 mL | 500 uL |
|---|---|---|---|
| 3A4 | 20 μM in K-buffer: | 20 μM in K-buffer: | 20 μM in K-buffer: |
| | substrate (40 μL) + | substrate (20 μL) + | substrate (10 μL) + |
| | K-buffer(1960 μL) | K-buffer(980 μL) | K-buffer(490 μL) |

TABLE 14
Inhibitory effect on CYP3A4

| | CYP3A4(M) |
|---|---|
| | inhibition |
| Compound No. | rate/1 μM |
| free base of Compound X | 23.8% |

It can be seen from Table 14 that free base of Compound X of the present invention have little inhibitory activity on CYP3A4.

### Example 14 Pharmaceutical Composition

Tablets of the crystal form A were prepared with the following components:

| | |
|---|---|
| Form A | 15 g |
| starch | 40 g |
| lactose | 37 g |
| PVP | 3 g |
| Sodium hydroxymethyl starch | 3 g |
| lauryl sodium sulfate | 1 g |
| magnesium stearate | 1 g |

By a conventional manner, the crystal form A and the starch are mixed and sieved, and then uniformly mixed with the other components described above, which was directly compressed.

### Example 15 Pharmaceutical Composition

Capsules of the crystal form I were prepared with the following components:

| | |
|---|---|
| Form I | 20 g |
| starch | 40 g |
| lactose | 32 g |
| PVP | 3 g |
| Sodium hydroxymethyl starch | 3 g |
| lauryl sodium sulfate | 1 g |
| magnesium stearate | 1 g |

By a conventional manner, the crystal form I and the starch are mixed and sieved, and then uniformly mixed with the other components described above, which was filled into ordinary transparent capsules.

All publications mentioned herein are incorporated by reference as if each individual document is cited as a reference, as in the present application. It should also be understood that, after reading the above teachings of the present invention, those skilled in the art can make various changes or modifications, equivalents of which falls in the scope of claims as defined in the appended claims.

What is claimed is:

1. A pharmaceutically acceptable salt of a compound of formula X, 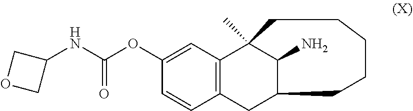 [SMILES: C[C@@]12CCCCC[C@@H](Cc3ccc(OC(=O)NC4COC4)cc31)[C@@H]2N]
a polymorph of the compound of formula X, or a polymorph of the pharmaceutically acceptable salt of the compound of formula X,
wherein the pharmaceutically acceptable salt is selected from the group consisting of hydrochloride, sulfate, hydrobromide, phosphate, methanesulfonate, maleate, L-tartrate, citrate, fumarate, succinate and besylate.

2. The pharmaceutically acceptable salt of the compound of formula X,
the polymorph of the compound of formula X, or the polymorph of the pharmaceutically acceptable salt of the compound of formula X of claim 1, wherein, the pharmaceutically acceptable salt is selected from the group consisting of sulfate, hydrobromide, phosphate, maleate, L-tartrate, fumarate and succinate.

3. The pharmaceutically acceptable salt of the compound of formula X,
the polymorph of the compound of formula X, or the polymorph of the pharmaceutically acceptable salt of the compound of formula X of claim 1, wherein, the polymorph is selected from the group consisting of:
A-type crystal of the maleate of the compound of formula X, i.e., crystal form A, the X-ray powder diffraction pattern of which has peaks at diffraction angles 2θ (°) of the following group A1: 7.75±0.2, 11.41±0.2, 13.03±0.2, 13.66±0.2, 15.10±0.2, 18.85±0.2, 21.49±0.2, 23.98±0.2 and 25.93±0.2;
B-type crystal of the sulfate of the compound of formula X, i.e., crystal form B, the X-ray powder diffraction pattern of which has peaks at diffraction angles 2θ (°) of the following group B1: 7.66±0.2, 13.57±0.2, 15.36±0.2, 18.01±0.2, 20.47±0.2, 21.02±0.2, 21.35±0.2, 23.17±0.2 and 31.05±0.2;
C-type crystal of the L-tartrate of the compound of formula X, i.e., crystal form C, the X-ray powder diffraction pattern of which has peaks at diffraction angles 2θ (°) of the following group 8.56±0.2, 11.68±0.2, 13.15±0.2, 15.37±0.2, 15.94±0.2, 16.99±0.2, 19.15±0.2, 22.42±0.2, 25.06±0.2 and 25.84±0.2;
D-1-type crystal of the phosphate of the compound of formula X, i.e., crystal form D-1, the X-ray powder diffraction pattern of which has peaks at diffraction angles 2θ (°) of the following group D-1-1: 4.30±0.2, 8.55±0.2, 12.79±0.2, 14.20±0.2, 15.61±0.2, 16.60±0.2, 17.17±0.2, 18.04±0.2, 20.74±0.2, 21.46±0.2, 22.36±0.2, 24.79±0.2, 25.51±0.2, 27.04±0.2 and 28.72±0.2;
D-2-type crystal of the phosphate of the compound of formula X, i.e., crystal form D-2, the X-ray powder diffraction pattern of which has peaks at diffraction angles 2θ (°) of the following group D-2-1: 4.31±0.2, 12.97±0.2, 14.11±0.2, 14.56±0.2, 15.14±0.2, 16.15±0.2, 17.26±0.2, 20.32±0.2, 21.85±0.2, 24.10±0.2 and 25.42±0.2;
E-type crystal of the hydrobromide of the compound of formula X, i.e., crystal form E, the X-ray powder diffraction pattern of which has peaks at diffraction angles 2θ (°) of the following group E1: 13.48±0.2, 13.83±0.2, 15.38±0.2, 17.28±0.2, 17.95±0.2, 19.67±0.2, 20.65±0.2, 22.31±0.2, 23.43±0.2, 24.78±0.2, 25.99±0.2, 27.11±0.2, 27.89±0.2, 31.08±0.2 and 31.59±0.2;
F-type crystal of the fumarate of the compound of formula X, i.e., crystal form F, the X-ray powder diffraction pattern of which has peaks at diffraction angles 2θ (°) of the following group F1: 13.61±0.2, 14.39±0.2, 14.84±0.2, 15.55±0.2, 17.70±0.2, 21.01±0.2, 22.54±0.2, 24.56±0.2 and 24.99±0.2;
G-1-type crystal of the succinate of the compound of formula X, i.e., crystal form G-1, the X-ray powder diffraction pattern of which has peaks at diffraction angles 2θ (°) of the following group G-1-1:10.78±0.2, 12.94±0.2, 14.47±0.2, 14.98±0.2, 15.31±0.2, 17.59±0.2, 19.63±0.2, 21.82±0.2, 22.57±0.2, 24.25±0.2, 25.29±0.2, 26.02±0.2 and 26.65±0.2;
G-2-type crystal of the succinate of the compound of formula X, i.e., crystal form G-2, the X-ray powder diffraction pattern of which has peaks at diffraction angles 2θ (°) of the following group G-2-1:10.89±0.2, 11.71±0.2, 13.06±0.2, 14.74±0.2, 15.37±0.2, 17.74±0.2, 18.58±0.2, 19.72±0.2, 20.56±0.2, 21.94±0.2, 22.21±0.2, 22.75±0.2, 24.94±0.2 and 26.14±0.2; and
crystal form I of the compound of formula X, the X-ray powder diffraction pattern of which has peaks at diffraction angles 2θ (°) of the following group I-1: 8.83±0.2, 11.51±0.2, 12.60±0.2, 13.13±0.2, 13.96±0.2, 15.93±0.2, 17.03±0.2, 19.78±0.2, 21.14±0.2, 22.06±0.2, 22.66±0.2, 23.19±0.2 and 25.07±0.2.

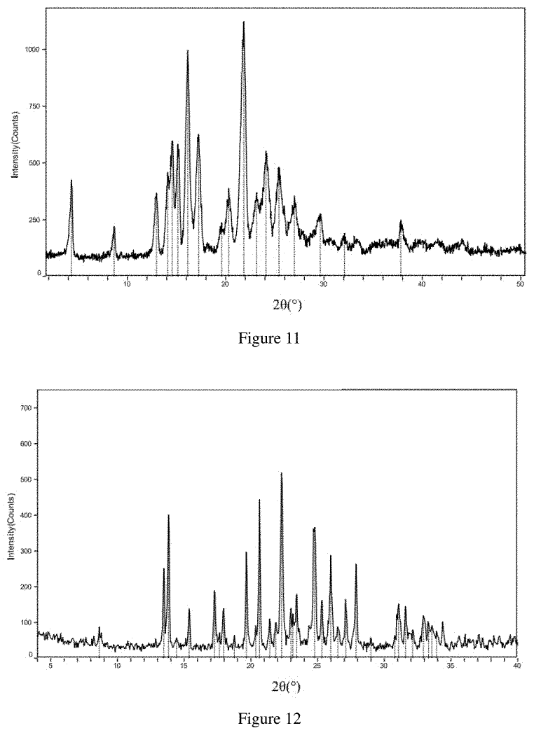

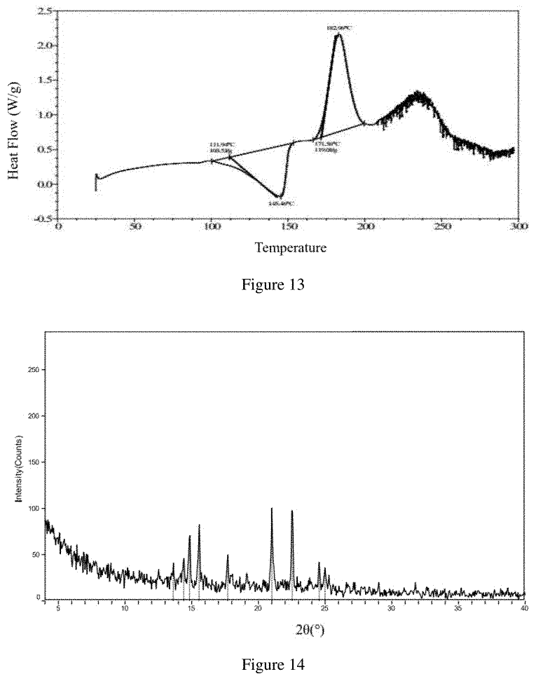

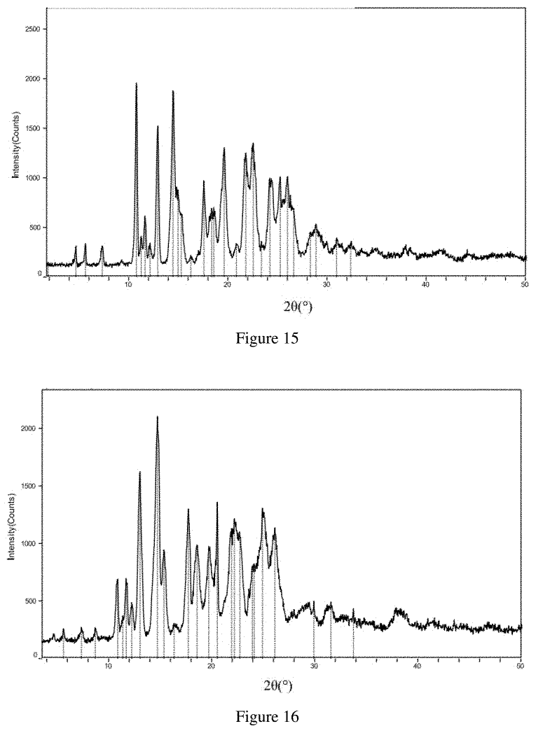

4. The pharmaceutically acceptable salt of the compound of formula X,
the polymorph of the compound of formula X, or the polymorph of the pharmaceutically acceptable salt of the compound of formula X of claim 3, wherein,
the X-ray powder diffraction pattern of the crystal form A is substantially as shown in FIG. 1;
the X-ray powder diffraction pattern of the crystal form B is substantially as shown in FIG. 4;
the X-ray powder diffraction pattern of the crystal form C is substantially as shown in FIG. 7;
the X-ray powder diffraction pattern of the crystal form D-1 is substantially as shown in FIG. 10;
the X-ray powder diffraction pattern of the crystal form D-2 is substantially as shown in FIG. 11;
the X-ray powder diffraction pattern of the crystal form E is substantially as shown in FIG. 12;
the X-ray powder diffraction pattern of the crystal form F is substantially as shown in FIG. 14;
the X-ray powder diffraction pattern of the crystal form G-1 is substantially as shown in FIG. 15;
the X-ray powder diffraction pattern of the crystal form G-2 is substantially as shown in FIG. 16.

5. The pharmaceutically acceptable salt of the compound of formula X,
the polymorph of the compound of formula X, or the polymorph of the pharmaceutically acceptable salt of the compound of formula X of claim 3, wherein, the X-ray powder diffraction pattern of the crystal form I is substantially as shown in FIG. 17.

6. A method of preparing a pharmaceutically acceptable salt of a compound of formula X, a polymorph of the compound of formula X, or a polymorph of the pharmaceutically acceptable salt of the compound of formula X, comprising steps of:
(1) deprotecting a compound X1 in a solvent to form the compound of formula X; and 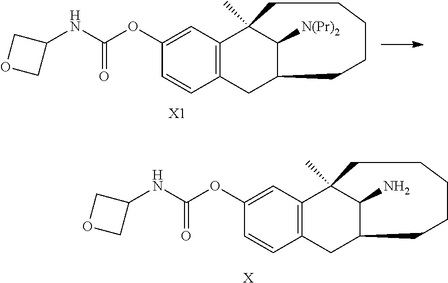
(2) optionally, conducting a salt forming reaction with the compound of formula X and an acid to form a pharmaceutically acceptable salt;
(3) optionally, crystallizing the compound of formula X or the pharmaceutically acceptable salt thereof formed by step (1) or step (2) to obtain a polymorph.

7. A pharmaceutical composition, comprising:
(a) the pharmaceutically acceptable salt of the compound of formula X, the polymorph of the compound of formula X, or the polymorph of the pharmaceutically acceptable salt of the compound of formula X of claim 1, and,
(b) a pharmaceutical acceptable carrier.

8. Use of the pharmaceutically acceptable salt of the compound of formula X, the polymorph of the compound of formula X, or the polymorph of the pharmaceutically acceptable salt of the compound of formula X of claim 1, in the preparation of a drug for the treatment of pain.

9. The use of claim 8, wherein, the pain is acute pain, chronic pain, postoperative pain, pain caused by neuralgia, pain caused by diabetic neuropathy, oral pain, pain associated with arthritis or osteoarthritis, or pain associated with cancer or its treatment.

10. A method of treating pain, comprising administering to a subject in need thereof a therapeutically effective amount of the pharmaceutically acceptable salt of the compound of formula X, the polymorph of the compound of formula X, or the polymorph of the pharmaceutically acceptable salt of the compound of formula X according to claim 1.

11. Use of the pharmaceutical composition of claim 7 in the preparation of a drug for the treatment of pain.

12. The use of claim 11, wherein, the pain is acute pain, chronic pain, postoperative pain, pain caused by neuralgia, pain caused by diabetic neuropathy, oral pain, pain associated with arthritis or osteoarthritis, or pain associated with cancer or its treatment.

13. A method of treating pain, comprising administering to a subject in need thereof a therapeutically effective amount of the pharmaceutical composition of claim 7.
